(19) United States
(12) Patent Application Publication
(10) Pub. No.: US 2022/0313710 A1
(43) Pub. Date: Oct. 6, 2022

(54) COMPOSITIONS AND METHODS USING NON-STEROIDAL ANTI-INFLAMMATORY DRUGS

(71) Applicant: APPLIED BIOLOGICAL LABORATORIES, INC., Brooklyn, NY (US)

(72) Inventor: Nazlie LATEFI, New York, NY (US)

(73) Assignee: APPLIED BIOLOGICAL LABORATORIES, INC., Brooklyn, NY (US)

(21) Appl. No.: 17/637,372

(22) PCT Filed: Aug. 22, 2020
(86) PCT No.: PCT/US2020/047551

§ 371 Date: Feb. 22, 2022

Related U.S. Application Data
(60) Provisional application No. 62/890,517, filed on Aug. 22, 2019.

(51) Int. Cl. A61K 31/616; A61K 38/40; A61K 38/47; A61K 47/10; A61K 47/38; A61K 36/886; A61K 9/00; A61P 11/04; A61J 7/00

(52) U.S. Cl. CPC A61K 31/616; A61K 38/40; A61K 38/47; A61K 47/10; A61K 47/38; A61K 36/886; A61K 9/006; A61P 11/04; A61J 7/0053

(57) ABSTRACT

Compositions comprising doses of NSAIDs less than 75 mg are provided. As shown herein, these compositions may be used for the treatment and prophylaxis of inflammatory conditions on the mucosa. In particular, acetyl salicylic acid at doses of less than 75 mg is shown to have therapeutic and prophylactic effect on the mucosa coupled without the adverse effects associated with NSAID administration.

### CROSS REFERENCE TO RELATED APPLICATIONS

The present application claims priority to U.S. App. No. 62/890,517, filed Aug. 22, 2019, which is hereby incorporated by reference in its entirety.

### BACKGROUND

Mucosal membranes are the epithelial membranes which line the oral cavity, the nasal, bronchial, pulmonary, trachea and pharynx airways, the optic and ophthalmic surfaces, the urogenital system, including the prostate, the reproductive system, and the gastrointestinal tract including the colon and rectal surfaces. Mucosal membranes represent the first portal of entry for many diseases. Mucosal membranes are also the subject of many disorders and diseases which are not strictly microbial in nature, for instance cystic fibrosis, prostatitis and digestive disorders. Particular problems arise in treating patients suffering from microbial infections, disorders or diseases of the mucosal membrane when the patient is allergic to a form of treatment such as an allergy to all or particular antibiotics.

These issues may cause inflammatory conditions to exist on the mucosal membranes resulting in pain of the subject infected. Typically, sore throat is characterized by pain, especially on swallowing, and is often accompanied by signs of inflammation of the larynx or pharynx. Pharyngitis (sore throat) is most commonly caused by viral infections such as the common cold, influenza, or mononucleosis. Less commonly, pharyngitis is caused by a bacterial infection. The most common bacterial infection of the throat is strep throat, which is caused by group A streptococcus. Rare causes of bacterial pharyngitis include gonorrhea, chlamydia, and Corynebacterium. Nearly all people will experience sore throats and providing a treatment regimen to help reduce inflammation associated with sore throats in a prophylactic and therapeutic modality would be of great benefit to this population.

Steroid therapy is often the therapy of choice for reducing inflammation (and sore throat) by the application of a topical corticosteroid to the affected area. In order to reduce systemic toxicity associated with steroid use, topical preparations were developed for inflammation disorders of the esophagus or gastrointestinal tract, such that the steroid could adhere to the esophageal mucosa and provide an anti-inflammatory effect. However, corticosteroid administration to the throat is often difficult to administer and control. Non-steroidal anti-inflammatory drugs (NSAIDs) such as ibuprofen, aspirin, and paracetamol have also been used to provide anti-inflammatory effects on sore throat as well, but have typically used at dosages greater than 325 mg. As shown in Moore et al, IJCP 56 (2002): 732-734, hereby incorporated by reference in its entirety, and particular in relation to adverse events in NSAID administration as shown, for example in Tables 2 and 3, NSAID administration is often plagued with adverse events associated therewith coupled with potentially inactivity.

### SUMMARY

In accordance with the foregoing objectives and others, the present disclosure provides pharmaceutical compositions, pharmaceutical products, methods of treatment of inflammatory conditions on mucosal membranes using non-steroidal anti-inflammatory drugs (NSAIDs) in a therapeutic window that is able to reduce inflammation and minimize adverse results.

Pharmaceutical compositions are provided which may comprise one or more pharmaceutically acceptable excipients, carriers, and/or diluents and one or more non-steroidal anti-inflammatory drugs (NSAID), wherein the total concentration of NSAIDs is less than (or from 0.01 to) 75 mg/ml. In certain embodiments, the one or more non-steroidal anti-inflammatory drugs comprise acetylsalicylic acid (aspirin). In certain embodiments, more than 90% of the NSAIDs in the composition is acetylsalicylic acid by weight of the composition.

The pharmaceutical composition may be in unit dose form (e.g. lozenge, capsule, caplet). In certain embodiments the unit dose form comprises less than (or from 0.01 to) 75 mg or less than 50 mg or less than 25 mg or less than 20 or less than 15 mg NSAIDs. In some embodiments, the lozenge comprises less than 20 mg (e.g., less than 10 mg, from 0.01 to 20 mg, from 0.01 to 15 mg, from 0.01 to 10 mg, from 0.1 to 10 mg) of acetylsalicylic acid. In some embodiments, more than 90% or more than 95% or more than 95% of the NSAIDs is acetylsalicylic acid by weight of the NSAIDs. For example, the lozenge may comprise:
a) from 1 to 10 mg of said one or more NSAIDs (e.g., acetylsalicylic acid);
b) optionally from 0.1 to 1 mg of lactoferrin;
c) optionally from 1 to 10 mg of lysozyme;
c) optionally from 10 to 100 mg of glycerol;
e) optionally from 100 to 400 mg of sweetener;
f) optionally from 1 to 20 mg menthol;
g) optionally from 1 to 20 mg carboxymethyl cellulose; and
h) optionally from 1 to 20 mg aloe.

The pharmaceutical composition may also be in the form of an oral spray. The oral spray may be formulated such that each spray administers less than (or from 0.01 mg to) 75 mg or less than 50 mg or less than 25 mg or less than 20 or less than 15 mg acetylsalicylic acid or less than 10 mg acetylsalicylic acid.

Pharmaceutical products comprising the oral spray is also within the scope of the present disclosure, wherein the pharmaceutical product may comprise:
(a) a body configured to be inserted into an oral passage for dispensing the oral spray composition;
(b) a reservoir in fluid communication with the orifice, wherein the oral spray composition is contained in the reservoir;
(c) a pump mechanism capable of expelling the oral spray composition through the orifice in appropriate sized aerosolized droplets; capable of coating the oral mucosa (e.g. mucosa of the throat) of a user.
   In some embodiments, the pump mechanism in the pharmaceutical product may be configured to expel from 100 μL to 1000 μL (e.g., 200 to 800 μL, 300 to 700 μL, 400 to 600 μL, 500 μL) of the oral spray composition. For example, each spray may comprise less than 75 mg or less than 50 mg or less than 25 mg or less than 20 or less than 15 mg NSAIDs. In some embodiments, the lozenge comprises less than 20 mg (e.g., less than 10 mg, from 0.01 to 20 mg, from 0.01 to 15 mg, from 0.01 to 10 mg, from 0.1 to 10 mg) of acetylsalicylic acid. In some embodiments, more than 90% or more than 95% or more than 99% of the NSAIDs is acetylsalicylic acid by weight of the NSAIDs. In some embodiments the oral spray composition may have a NSAID concentration of less than (or from 0.01 mg/ml to) 100 mg/ml (e.g., less than 75 mg/ml, less than 50 mg/ml, less than 25 mg/ml, less than 10 mg/ml, from 0.01 to 100 mg/ml, from 0.1 mg/ml to 100 mg/ml, from 1 mg/ml to 100 mg/ml, from 0.01 to 75 mg/ml, from 0.1 mg/ml to 75 mg/ml, from 1 mg/ml to 75 mg/ml, from 0.01 to 50 mg/ml, from 0.1 mg/ml to 50 mg/ml, from 1 mg/ml to 50 mg/ml, from 0.01 to 25 mg/ml, from 0.1 mg/ml to 25 mg/ml, from 1 mg/ml to 25 mg/ml, from 0.01 to 10 mg/ml, from 0.1 mg/ml to 10 mg/ml, from 1 mg/ml to 10 mg/ml).

Methods for the treatment or prophylaxis of sore throat in a subject in need thereof are also disclosed here, wherein the method comprises administration of these pharmaceutical composition to the subject in need thereof. In some embodiments, the method for the treatment or prophylaxis of sore throat in a subject in need thereof may comprise administering less than 75 mg (e.g., less than 50 mg, less than 25 mg, less than 15 mg) of a plurality of NSAIDs to said throat. The dosage regimens described herein allow for anti-inflammatory responses to be produced on the mucosa of the user thereby treating and/or preventing the anti-inflammatory response that would have been present without such administration. For example, less than 325 mg (e.g., less than 100 mg, less than 75 mg, less than 50 mg, less than 25 mg, less than 20 mg, less than 15 mg) of the plurality of NSAIDs are administered in 24 hours. In some embodiments, less than 325 mg (e.g., less than 100 mg, less than 75 mg, less than 50 mg, less than 25 mg, less than 20 mg, less than 15 mg) of the plurality of NSAIDs are administered in 12 hours.

In certain implementations, the pharmaceutical composition comprises:
a) from 1 to 10 mg/ml of said one or more NSAIDs;
b) optionally from 0.1 to 1 mg/ml of lactoferrin;
c) optionally from 1 to 10 mg/ml of lysozyme;
c) optionally from 10 to 100 mg/ml of glycerol;
e) optionally from 100 to 400 mg/ml of sweetener;
f) optionally from 1 to 20 mg/ml menthol;
g) optionally from 1 to 20 mg/ml carboxymethyl cellulose; and
h) optionally from 1 to 20 mg/ml aloe.

### BRIEF DESCRIPTION OF FIGURES

FIG. 1A is shows the prostaglandin E2 (PGE-2) production taken from 3D in vitro respiratory epithelia treated with the indicated test condition when NSAID compositions were added after initiation of the inflammatory condition. FIG. 1B compares the results of the 0.6 mg/ml acetylsalicylic acid (ASA) condition to the positive control. Increased “*” denotes increased statistical significance between measurements (i.e., *:=p-value <0.05; **:=p-value:=0.01; ***:=p-value <0.001) and error bars represent the standard deviation of measurements.

FIG. 2A shows the prostaglandin E2 production taken from 3D in vitro respiratory epithelia treated with the indicated test condition when NSAID compositions were added before initiation of the inflammatory condition. FIG. 2B compares the results of the 0.6 mg/ml acetylsalicylic acid (ASA) condition to the positive control. Increased “*” denotes increased statistical significance between measurements (i.e., *:=p-value <0.05; **:=p-value:=0.01; ***:=p-value <0.001) and error bars represent the standard deviation of measurements.

FIG. 3A illustrates the TEER measurements on the 3D in vitro respiratory epithelia following each test condition of administration after initiation of the inflammatory conditions. FIG. 3B shows the TEER measurements on the 3D in vitro respiratory epithelia following each test condition of administration before the initiation of inflammatory conditions. Increased “*” denotes increased statistical significance between measurements (i.e., *:=p-value <0.05; **:=p-value:=0.01; ***:=p-value <0.001) and error bars represent the standard deviation of measurements.

FIG. 4A shows the interleukin-8 (IL-8) production taken from 3D in vitro respiratory epithelia treated with the indicated test condition when NSAID compositions were added after initiation of the inflammatory condition. FIG. 4B shows the interleukin-8 production taken from 3D in vitro respiratory epithelia treated with the indicated test condition when NSAID compositions were added after initiation of the inflammatory condition. Increased “*” denotes increased statistical significance between measurements (i.e., *:=p-value <0.05; **:=p-value:=0.01; ***:=p-value <0.001) and error bars represent the standard deviation of measurements.

FIG. 5A shows the cytotoxicity of A549 cells as measured by LDH release in several test conditions. FIG. 5B shows the interleukin-8 (IL-8) production of A549 cells in several test conditions. FIG. 5C shows the PGE-2 production of A549 cells in several test conditions.

FIG. 6A shows the PGE-2 production following different bradykinin applications to two different 3D in vitro respiratory models. FIG. 6B shows the IL-8 production at 24 hours for each of these models. FIG. 6C shows the TEER for each of the models at 24 hours. FIG. 6D shows the cytotoxicity as measured by LDH for each of these models at 24 hours. FIG. 6E shows the IL-8 production at 48 hours for each of these models. FIG. 6F shows the TEER for each of the models at 48 hours. FIG. 6G shows the cytotoxicity as measured by LDH for each of these models at 48 hours.

FIG. 7A shows the PGE-2 production following different bradykinin applications to MucilAir 3D in vitro models. FIG. 7B shows the TEER at 24 hours for each of these models. FIG. 7C shows the TEER at 48 hours. FIG. 7D shows the IL-8 production at 24 hours for each of these models. FIG. 7E shows the IL-8 production at 24 hours for each of these models.

### DETAILED DESCRIPTION

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive.

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. The exact values of all half integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, and from 0.1% to 2.5%. It will be understood that the sum of all weight % of individual components will not exceed 100%.

Unless otherwise specified, an indicated percentage is intended to be a weight by weight (w/w) percentage. However, other compositional percentages may be indicated, such as weight/volume (w/v) which, unless otherwise specified, given in g/100 mL. For example, a weight percentage of 0.6% (w/v) is 6 mg/ml.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight. The use of“comprise” is intended to expressly disclose the “consist essentially” and “consist” embodiments.

The term “pharmaceutical composition,” as used herein, represents a composition containing a compound described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gel cap, lozenge). In certain embodiments, the pharmaceutical composition is formulated is a spray (e.g., an oral spray), or a lozenge.

The pharmaceutical compositions of the present disclosure are suitable for treating inflammatory conditions of the gastrointestinal tract, for example inflammatory conditions of the upper gastrointestinal tract

As used herein, the phrase “pharmaceutically acceptable” indicates that the specified material is generally safe for ingestion or contact with biologic tissues at the levels employed. Pharmaceutically acceptable is used interchangeably with physiologically compatible. It will be understood that the pharmaceutical compositions of the disclosure include nutraceutical compositions (e.g., dietary supplements) unless otherwise specified.

Useful pharmaceutical carriers, excipients, and diluents for the preparation of the compositions hereof, can be solids, liquids, or gases. These include any and all solvents, dispersion media, coatings, antibacterial and antifungal agents, isotonic and absorption delaying agents and the like. The pharmaceutically acceptable carrier or excipient does not destroy the pharmacological activity of the disclosed compound and is nontoxic when administered in doses sufficient to deliver a therapeutic amount of the compound. Thus, the compositions can take the form of tablets, pills, capsules, suppositories, powders, enterically coated or other protected formulations (e.g., binding on ion-exchange resins or packaging in lipid-protein vesicles), sustained release formulations, solutions, suspensions, elixirs, and aerosols. The carrier can be selected from the various oils including those of petroleum, animal, vegetable or synthetic origin, e.g., peanut oil, soybean oil, mineral oil, and sesame oil. Water, saline, aqueous dextrose, and glycols are examples of liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, chitosan, talc, glucose, lactose, gelatin, malt, rice, flour, chalk, silica, magnesium stearate, sodium stearate, glycerol monostearate, sodium chloride, dried skim milk, glycerol, propylene glycol, water, and ethanol. The compositions may be subjected to conventional pharmaceutical additives such as preservatives, stabilizing agents, wetting or emulsifying agents, salts for adjusting osmotic pressure, and buffers. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for administration to the recipient.

Non-limiting examples of pharmaceutically acceptable carriers and excipients include sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols, such as polyethylene glycol and propylene glycol; esters such as ethyl oleate and ethyl laurate; agar; buffering agents such as magnesium hydroxide and aluminum hydroxide; alginic acid; isotonic saline; Ringer's solution; ethyl alcohol; phosphate buffer solutions; non-toxic compatible lubricants such as sodium lauryl sulfate and magnesium stearate; coloring agents; releasing agents; coating agents; sweetening, flavoring and perfuming agents; preservatives; antioxidants; ion exchangers; alumina; aluminum stearate; lecithin; self-emulsifying drug delivery systems (SEDDS) such as d-atocopherol polyethyleneglycol 1000 succinate; surfactants used in pharmaceutical dosage forms such as Tweens or other similar polymeric delivery matrices; serum proteins such as human serum albumin; glycine; sorbic acid; potassium sorbate; partial glyceride mixtures of saturated vegetable fatty acids; water, salts or electrolytes such as protamine sulfate, disodium hydrogen phosphate, potassium hydrogen phosphate, sodium chloride, and zinc salts; colloidal silica; magnesium trisilicate; polyvinyl pyrrolidone; cellulose-based substances; polyacrylates; waxes; and polyethylene-polyoxypropylene-block polymers. Cyclodextrins such as α-, β-, and γ-cyclodextrin, or chemically modified derivatives such as hydroxyalkylcyclodextrins, including 2- and 3-hydroxypropyl-cyclodextrins, or other solubilized derivatives can also be used to enhance delivery of the compounds described herein.

The compounds described herein may be present as a pharmaceutically acceptable salt. Typically, salts are composed of a related number of cations and anions (at least one of which is formed from the compounds described herein) coupled together (e.g., the pairs may be bonded ionically) such that the salt is electrically neutral. Pharmaceutically acceptable salts may retain or have similar activity to the parent compound (e.g., an ED₅₀ within 10%) and have a toxicity profile within a range that affords utility in pharmaceutical compositions. For example, pharmaceutically acceptable salts may be suitable for use in contact with the tissues of humans and animals without undue toxicity, irritation, allergic response and are commensurate with a reasonable benefit/risk ratio. Pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, dichloroacetate, digluconate, dodecylsulfate, ethanesulfonate, formate, fumarate, glucoheptonate, glutamate, glycerophosphate, hemisulfate, heptonate, hexanoate, hippurate, hydrobromide, hydrochloride, hydroiodide, 2-hydroxy-ethanesulfonate, isethionate, lactobionate, lactate, laurate, lauryl sulfate, malate, maleate, malonate, mandelate, methanesulfonate, mucate, 2-naphthalenesulfonate, nicotinate, nitrate, oleate, oxalate, palmitate, pamoate, pantothenate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative basic salts include alkali or alkaline earth metal salts include sodium, lithium, potassium, calcium, and magnesium, aluminum salts, as well as nontoxic ammonium, quaternary ammonium, and amine cations, including, but not limited to ammonium, tetramethylammonium, tetraethylammonium, methylamine, dimethylamine, trimethylamine, triethylamine, caffeine, and ethylamine.

Pharmaceutically acceptable acid addition salts of the disclosure can be formed by the reaction of a compound of the disclosure with an equimolar or excess amount of acid. Alternatively, hemi-salts can be formed by the reaction of a compound of the disclosure with the desired acid in a 2:1 ratio, compound to acid. The reactants are generally combined in a mutual solvent such as diethyl ether, tetrahydrofuran, methanol, ethanol, iso-propanol, benzene, or the like. The salts normally precipitate out of solution within, e.g., one hour to ten days and can be isolated by filtration or other conventional methods.

Unit dosage forms, also referred to as unitary dosage forms, often denote those forms of medication supplied in a manner that does not require further weighing or measuring to provide the dosage (e.g., tablet, capsule, caplet). For example, a unit dosage form may refer to a physically discrete unit suitable as a unitary dosage for human subjects and other mammals, each unit containing a predetermined quantity of active material calculated to produce the desired therapeutic effect, in association with any suitable pharmaceutical excipient or excipients. Exemplary, non-limiting unit dosage forms include a tablet (e.g., a chewable tablet), caplet, capsule (e.g., a hard capsule or a soft capsule), lozenge, film, strip, and gel cap. In certain embodiments, the compounds described herein, including crystallized forms, polymorphs, and solvates thereof, may be present in a unit dosage form.

The term “effective amount” or “therapeutically effective amount” of an agent (e.g. acetylsalicylic acid), as used herein, is that amount sufficient to effect beneficial or desired results, such as clinical results, and, as such, an “effective amount” depends upon the context in which it is being applied. In some embodiments, the compounds are administered in an effective amount for the treatment or prophylaxis of a disease disorder or condition. In another embodiment, in the context of administering an agent that is an anti-inflammatory agent, an effective amount of an agent is, for example, an amount sufficient to achieve alleviation or amelioration or prevention or prophylaxis of one or more symptoms or conditions such as sore throat, as compared to the response obtained without administration of the agent.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. The term “prevent” or “prophylaxis” as used herein, includes delaying the onset of or progression of a disease or physiological manifestation of disease. The term “treat” includes reducing, diminishing, eliminating, ameliorating, forestalling, slowing the progression of, and/or delaying the onset of a given disease or physiological manifestation thereof.

Typically, the treatment of a condition (e.g., the inflammatory conditions described herein such as sore throat) is an approach for obtaining beneficial or desired results including clinical results. Inflammation often occurs when tissues are injured by viruses, bacteria, trauma, chemicals, heat, cold, allergens, or any other harmful stimulus. Chemicals including bradykinin, histamine, serotonin and others are released, attracting tissue macrophages and white blood cells to localize in an area to engulf and destroy foreign substances. During this process, chemical mediators such as TNFα are released, giving rise to inflammation. Inflammatory disorders are those in which the inflammation is sustained or chronic. Beneficial or desired results to an inflammatory disease, condition, or disorder can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

As used herein, the term “subject” refers to any organism to which a composition and/or compound in accordance with the disclosure may be administered, e.g., for experimental, diagnostic, prophylactic, and/or therapeutic purposes. Typical subjects include any animal (e.g., mammals such as mice, rats, rabbits, non-human primates, and humans). A subject in need thereof is typically a subject for whom it is desirable to treat a disease, disorder, or condition as described herein. For example, a subject in need thereof may seek or be in need of treatment, require treatment, be receiving treatment, may be receiving treatment in the future, or a human or animal that is under care by a trained professional for a particular disease, disorder, or condition.

The identification of a particular active agent as having a certain activity is not limiting, unless otherwise indicated, and does not preclude the same agent from having additional activities.

Pharmaceutical compositions are provided which may comprise one or more pharmaceutically acceptable excipients, carriers, and/or diluents and one or more non-steroidal anti-inflammatory drugs (NSAID), wherein the total concentration of NSAIDs is less than 75 mg/ml. In certain embodiments, the one or more non-steroidal anti-inflammatory drugs comprise acetylsalicylic acid (aspirin). In certain embodiments, more than 90% of the NSAIDs in the composition is acetylsalicylic acid by weight of the composition.

The pharmaceutical composition may be in unit dose form (e.g. lozenge, capsule, caplet). In certain embodiments the unit dose for comprises less than 75 mg or less than 50 mg or less than 25 mg or less than 20 or less than 15 mg acetylsalicylic acid.

The pharmaceutical composition may also be in the form of an oral spray. The oral spray may be formulated such that each spray administers less than 75 mg or less than 50 mg or less than 25 mg or less than 20 or less than 15 mg acetylsalicylic acid or less than 10 mg acetylsalicylic acid.

The one or more NSAIDs may comprise acetylsalicylic acid (ASA). The NSAID may be, for example a COX-2 inhibitor or a COX-1 inhibitor. An NSAID for use with the present disclosure may be NS-398 (N-[2-(cyclohexyloxy)-4-nitrophenyl]methanesulfonamide), ASA, celoxcoxib or tilmacoxib. In some embodiments, the concentration of the acetylsalicylic acid is less than 10 mg/ml. In some embodiments, the total concentration of NSAIDs is less than 20 mg/ml. In some embodiments, the total NSAID content of the composition is more than 90% or more than 95% or more than 99% acetylsalicylic acid by weight of the NSAID content. In various implementations, acetylsalicylic acid is the only NSAID in the composition or more than 90% or more than 95% or more than 99% of the NSAIDs is acetylsalicylic acid by weight of the NSAIDs in the composition. In certain embodiments, acetylsalicylic acid and aloe extract are the only NSAIDs in the composition or more than 90% or more than 95% or more than 99% of the NSAIDs is acetylsalicylic acid and aloe extract by weight of the NSAIDs in the composition. The acetylsalicylic acid and aloe extract may be present in the composition with a weight ratio of from 100:1 to 1:100 or from 100:1 to 50:1 or from 50:1 from 10:1 or from 10:1 to 1:10 or from 5:1 to 1:5 or from 2:1 to 1:2 or from 10:1 to 1:1 or from 5:1 to 1:1 or from 2:1 to 1:1 or from 1:1 to 1:10 or from 1:1 to 1:5 or from 1:1 to 1:2 or from 1:10 to 1:50 or from 1:50 to 1:100. In various implementations, the total weight of the one or more NSAIDs is less than 50 mg (e.g., less than 25 mg, less than 20 mg, less than 10 mg).

The pharmaceutical compositions may provide an anti-inflammatory effect which may be a reduction in one or more of the symptoms of erythema (redness), edema (swelling), pain and pruritus which are characteristic of inflammatory conditions of mucosal membranes. Typically, the pharmaceutical compositions may be used for the treatment of pharyngitis (sore throat). The pharyngitis may be characterized by pain and swelling in the pharynx. Pharyngitis is commonly caused by bacterial (e.g., Streptococcal) or viral infection, and may be treated with topical compositions comprising acetylsalicylic acid as described.

In certain embodiments, the dosage form can be administered, for example, 1×, 2×, 3×, 4×, 5×, 6×, 7×, or 8×, per day. One or more dosage form can be administered, for example, for 1, 2, 3, 4, 5, 6, 7 days, or even longer. One or more dosage forms can be administered, for example, for 1, 2, 3, 4 weeks, or even longer. One or more dosage forms can be administered, for example, for 1, 2, 3, 4, 5, 6, 7, 8, 9, 10, 11, 12 months, or even longer. One or more dosage forms can be administered until the patient, subject, mammal, mammal in need thereof, human, or human in need thereof, does not require treatment, prophylaxis, or amelioration of any disease or condition such as, for example, inflammatory conditions such as sore throat. In some aspects, the dosage form may be co-administered with other pharmaceutical compositions until the patient, subject, mammal, mammal in need thereof, human, or human in need thereof, does not require treatment, prophylaxis, or amelioration of any disease or condition including inflammation or pain. In various implementations, no more than 300 mg (e.g., no more than 200 mg, no more than 100 mg, no more than 50 mg, no more than 25 mg) of acetylsalicylic acid (ASA) is administered in a 24-hour period. In some embodiments, no more than 300 mg of acetylsalicylic acid (e.g., no more than 200 mg, no more than 100 mg, no more than 50 mg, no more than 25 mg) of acetylsalicylic acid (ASA) is administered in a 12-hour period.

A sore throat is pain, scratchiness or irritation of the throat that. The most common cause of a sore throat (pharyngitis) is a viral infection, such as a cold or the flu. A sore throat caused by a virus usually resolves on its own, although time to recovery can take a week or more and be an uncomfortable process.

Several ingredients may be present in an amount to mimic the rheology of mucous. In some embodiments, the pharmaceutical compositions (or the compositions when administered to mucosa) are capable of mimic the rheological parameters of the mucous secreted from that mucosa (e.g., oral and/or nasal mucosa). Administered formulations with similar rheological parameters to mucous may several beneficial effects for the formulations. For example, without wishing to be bound by theory, by mimicking the complex rheological properties of mucous, the residence time for various pathogens may be increased prior to contact with the surface of the membranes resulting in increased targeted binding and/or anti-microbial effect and/or the barrier function of the mucous in combination with the pharmaceutical composition may be increased. Rheological parameters are described in, for example, Lai, S. Et al., Adv. Drug. Deliv. Rev. 61 (2009): 86-100, hereby incorporated by reference in its entirety. In some embodiments, the pharmaceutical compositions have non-Newtonian rheology in formulation and following administration to mucosa (e.g., the administered formulation may be a non-Newtonian gel). In some embodiments, the pharmaceutical composition may have a Viscosity of between 10⁻³ and 10² Pa·s (e.g., between 10⁻¹ Pa·s and 10⁻² Pa·s, between 10⁻² Pa·s and 10⁻¹ Pa·s, between 10⁻¹ Pa·s and 1 Pa·s, between 1 Pa·s and 10 Pa·s, between 10 Pa·s and 10² Pa·s) at a shear rate of 10 Hz at 25° C. Various agents may be used to mimic the rheology of mucous. For example, the pharmaceutical composition may comprise one or more agents selected from mucins, plant mucilage, marshmallow extract, lysozyme, and/or lactoferrin (e.g., apolactoferrin) in amounts capable of mimicking the rheology of mucous. In some embodiments, the pharmaceutical compositions may comprise mucins. In some embodiments, the pharmaceutical compositions may comprise plant mucilage. In some embodiments, the pharmaceutical compositions may comprise mucins and plant mucilage. In some embodiments, the pharmaceutical compositions may comprise mucins and marshmallow extract. In some embodiments, the pharmaceutical composition may comprise plant mucilage and marshmallow extract. In some embodiments, the pharmaceutical composition may comprise mucins, marshmallow extract, and plant mucilage.

In certain embodiments, the pharmaceutical composition may comprise an amount of ingredient that effects the rheology of the material deposited on the mucous membranes. For example, the pharmaceutical composition may comprise lactoferrin (e.g., apolactoferrin) and/or lysozyme. In certain implementations, the composition comprises from 0.01% (w/v) to 10% (w/v) or from 0.1% (w/v) to 10% (w/v) or from 0.1% (w/v) to 5% (w/v) or from 0.01% (w/v) to 5% (w/v) or from 0.1% (w/v) to 1% (w/v) lactoferrin and/or lysozyme. The weight ratio of lactoferrin:lysozyme may be, for example, 100:1 to 1:100 (e.g., 1:1 to 1:100, 1:1 to 1:50, 1:1 to 1:20, 1:5 to 1:15, 100:1 to 1:1, 50:1 to 1:1, 20:1 to 1:1, 15:1 to 1:1, 50:1 to 1:50, 20:1 to 1:20, 15:1 to 1:15, 10:1 to 1:10). In certain embodiments, the lactoferrin and/or lysozyme in combination with the NSAIDs (e.g., salicylate and derivatives thereof including salicylic acid and acetylsalicylic acid) may increase the anti-inflammatory response as compared to the combined results of an otherwise identical composition without the NSAIDs and an otherwise identical composition without the lactoferrin and lysozyme. For example, the NSAIDs in combination with the lactoferrin and/or lysozyme may provide decreased prostaglandin (e.g., PGE2) production and/or decreased interleukin-8 (IL-8) production as compared to the combined result of the NSAIDs alone (e.g., in aqueous buffer, in the pharmaceutical compositions disclosed herein) and the lactoferrin and lysozyme (e.g., in aqueous buffer, in the pharmaceutical compositions disclosed herein).

The present disclosure is partially premised on the discovery that doses of certain NSAIDs have are able to decrease the inflammatory response to certain cytokines (e.g., bradykinin and concomitant production of prostaglandin) and maintain barrier integrity of mucosal membranes. Furthermore, these same NSAID has minimal effect on the inflammatory response (e.g., prostaglandin production) of mucosal membranes and is coupled to significant degradation of barrier integrity. For example, acetylsalicylic acid, which is typically administered at doses of greater than 75 mg (e.g., baby aspirin) and up to 325 mg, has is able to inhibit the inflammatory response at doses less than 75 mg or less than 70 mg or less than 65 mg or less than 60 mg or less than 55 mg or less than 50 mg or less than 45 mg or less than 40 mg or less than 35 mg or less than 30 mg or less than 25 mg or less than 20 mg or less than 15 mg. Furthermore, this same dosage may have no effect on membrane integrity, particularly when administered at concentration less than 30 mg/ml (e.g., less than 20 mg/ml, less than 15 mg/ml, from 0.1 to 30 mg/ml, from 1 to 15 mg/ml, from 1 to 10 mg/ml). Administration may occur more than once (e.g., two times, three times) in a period greater than 10 minutes (e.g., greater than 15 minutes). In certain embodiments, the NSAID (e.g., acetylsalicylic acid) is administered such that that in 24 hours, less than 75 mg or less than 70 mg or less than 65 mg or less than 60 mg or less than 55 mg or less than 50 mg or less than 45 mg or less than 40 mg or less than 35 mg or less than 30 mg or less than 25 mg or less than 20 mg or less than 15 mg is administered. In certain embodiments, the NSAID (e.g., acetylsalicylic acid) is administered such that that in 12 hours, less than 75 mg or less than 70 mg or less than 65 mg or less than 60 mg or less than 55 mg or less than 50 mg or less than 45 mg or less than 40 mg or less than 35 mg or less than 30 mg or less than 25 mg or less than 20 mg or less than 15 mg is administered.

In certain implementations, the pharmaceutical composition comprises:
a) from 1 to 10 mg/ml of the one or more NSAIDs;
b) optionally from 0.1 to 1 mg/ml of lactoferrin;
c) optionally from 1 to 10 mg/ml of lysozyme;
c) optionally from 10 to 100 mg/ml of glycerol;
e) optionally from 100 to 400 mg/ml of sweetener;
f) optionally from 1 to 20 mg/ml menthol;
g) optionally from 1 to 20 mg/ml carboxymethyl cellulose; and
h) optionally from 1 to 20 mg/ml aloe.
   In some embodiments, the pharmaceutical composition is in an aqueous carrier such as isotonic saline or 0.9% NaCl, 1.25 mM CaCl₂), and 10 mM 4-(2-hydroxyethyl)-1-piperazineethanesulfonic acid (HEPES).

The NSAID (e.g., salicylate and derivative thereof such as salicylic acid and acetylsalicylic acid) may be administered such that the inflammatory response is minimized (or removed) and/or the barrier function of the mucosa (e.g., as measured by TEER) is not decreased. As shown herein, such a response may only be possible within a certain therapeutic window. In some embodiments, the NSAID may be administered such that the inflammatory response (e.g., as measured by prostaglandin E2 production) is minimized (or removed) and/or the barrier function of the mucosa (e.g., as measured by TEER) is not decreased. For example, less than 325 mg (e.g., less than 300 mg, less than 250 mg, less than 200 mg, less than 150 mg, less than 100 mg, less than 50 mg, less than 25 mg, less than 10 mg) may be administered daily to the mucosal membranes such as those of the throat. In some embodiments, less than 325 mg (e.g., less than 300 mg, less than 250 mg, less than 200 mg, less than 150 mg, less than 100 mg, less than 50 mg, less than 25 mg, less than 10 mg) may be administered twice daily (e.g., every 12 hours) to the mucosal membranes such as those of the throat. The pharmaceutical composition may be formulated with a concentration of NSAID such as acetylsalicylic acid to achieve a concentration on the mucosal membrane appropriate for the presently described dosage window accounting for various dilution factors that may occur following administration. In some embodiments, the pharmaceutical composition may have a concentration of less than 325 mg/ml (e.g., less than 300 mg/ml, less than 250 mg/ml, less than 200 mg/ml, less than 150 mg/ml, less than 100 mg/ml, less than 50 mg/ml, less than 25 mg/ml, less than 10 mg/ml).

A therapeutic compound disclosed herein may be a non-steroidal anti-inflammatory drug (NSAID). Typically, NSAIDs reduce inflammation by blocking cyclooxygenase. NSAIDs include, without limitation, NSAIDs may be classified based on their chemical structure or mechanism of action. The present disclosure is partially premised on the discovery that NSAIDs are capable of downregulating prostaglandin production by inhibiting cyclooxygenase (COX) enzymes such as COX-1, COX-2 and/or nuclear factor kappa-light-chain-enhancer of activated B cells (NF-κB) protein. Non-limiting examples of NSAIDs include a non-selective cyclo-oxygenase (COX) inhibitor, a selective cyclooxygenase 1 (COX 1) inhibitor, and a selective cyclooxygenase 2 (COX 2) inhibitor. For example, the NSAID may be NS-398, salicylate or a salicylate derivative. Examples of a suitable salicylate derivative NSAID include, without limitation, salicylic acid, acetylsalicylic acid (also referred to as aspirin or ASA), diflunisal, and salsalate. In particular embodiments, the NSAID is the sodium or potassium salt of acetylsalicylic acid. Examples of a suitable p-amino phenol derivative NSAID include, without limitation, paracetamol and phenacetin. Examples of a suitable propionic acid derivative NSAID include (often referred to as profens), without limitation, alminoprofen, benoxaprofen, dexketoprofen, fenoprofen, flurbiprofen, ibuprofen, indoprofen, ketoprofen, loxoprofen, naproxen, oxaprozin, pranoprofen, and suprofen. Examples of a suitable acetic acid derivative NSAID include, without limitation, aceclofenac, acemetacin, actarit, alcofenac, amfenac, clometacin, diclofenac, etodolac, felbinac, fenclofenac, indometacin, ketorolac, metiazinic acid, mofezolac, nabumetone, naproxen, oxametacin, sulindac, and zomepirac. Examples of a suitable enolic acid (Oxicam) derivative NSAID include, without limitation, droxicam, isoxicam, lornoxicam, meloxicam, piroxicam, and tenoxicam. Examples of a suitable fenamic acid derivative NSAID include, without limitation, flufenamic acid, mefenamic acid, meclofenamic acid, and tolfenamic acid. In certain embodiments, the only NSAID in the composition is a salicylate derivative or the composition comprises less than 1% or less than 0.5% or less than 0.1% of NSAIDs other than salicylate derivatives by weight of the composition. In some embodiments, the total NSAIDs concentration in the pharmaceutical composition is than 120 mg/ml or less than 100 mg/ml or less than 75 mg/ml or less than 50 mg/ml or less than 30 mg/ml or less than 20 mg/ml. In some embodiments, the total NSAIDs content in the pharmaceutical composition is than 15% or less than 10% by weight of the composition.

The composition may comprise the one or more NSAIDs (e.g., salicylate derivatives such as acetylsalicylic acid) dispersed in a carrier, and typically, but not necessarily, a liquid carrier. The liquid carrier is ideally, but not necessarily, of suitable rheology to be sprayed as an aerosol or fine mist. The composition may comprise one or more ingredients selected from the group consisting of an emollient, an occlusive, a humectant, a carrier, an excipient, an emulsifier, and an essential oil. Typically, excipients, carriers and/or diluents should be compatible with the human mucosa and epithelium, and should not cause excessive drying or irritation to the mucosa or epithelium. The excipients should also account for the fact that water will tend to evaporate at body temperature and as such a secondary solvent may be included to aid in maintaining the soluble components in solution. The carrier may include a polyol, such as a C₂-C₈ polyol, including without limitation, glycerol, propylene glycol, 1,3-propane diol, butylene glycol, 1,4-butane diol, erythritol, threitol, arabitol, xylitol, mannitol, sorbitol, pentylene glycol, hexylene glycol, caprylyl glycol, hydrogenated starch hydrolysates, isomalt, maltitol, and the like. The compositions may comprise an amount of an alcohol, such as ethanol, provided it is in an amount that does not irritate or dry the mucosa or any drying or irritation which may occur is offset by other ingredients. In some embodiments, the compositions are free of alcohol (e.g., ethanol). In one embodiment, the carrier is an aqueous carrier including from 1-95% or from 5-50% or from 10-40% or from 15-35% or from 20-30% 1,3-propanediol, on a (v/v), (w/v), or (w/w) basis. In some embodiments, the composition may have a kinematic viscosity ranging from 1-1,500 or from 5-1,000 or from 10-750 or from 20-500 centiStokes (mm²/s). The compositions may have a Newtonian or non-Newtonian rheology. The compositions may be, for example, shear thinning and/or thixotropic, such that they readily flow through a spray nozzle and form a mist of suitable droplet size on shearing, but thicken in situ to form a film on the mucosa which is resistant to clearance from the nasal and/or oral cavity such that the active remain on the mucosa for a time sufficient to neutralize pathogens in contact with the mucosa. Typically, the composition will be of suitable viscosity to possess a residence time on the mucosa of the nasal and/or oral cavities of at least 1 minute, such as, at least 5, 10, 15, 20, 25, or 30 minutes following application.

For example, the pharmaceutical composition may have a weight ratio of rheology modifying compounds (e.g., lactoferrin, lysozyme) to NSAID (e.g., salicylate derivative such as acetylsalicylic acid) of from 10:1 to 1:10 (e.g., 8:1 to 1:8, 6:1 to 1:6, 2:1 to 1:2, 3:2 to 2:3). In some embodiments, the pharmaceutical composition may have a weight ratio of lactoferrin and lysozyme to NSAID of from 10:1 to 1:10 (e.g., 8:1 to 1:8, 6:1 to 1:6, 2:1 to 1:2, 3:2 to 2:3). In various implementations, the pharmaceutical composition may have a weight ratio of lactoferrin and lysozyme to acetylsalicylic acid of from 10:1 to 1:10 (e.g., 8:1 to 1:8, 6:1 to 1:6, 2:1 to 1:2, 3:2 to 2:3). In some embodiments, the total NSAID concentration (e.g., the acetylsalicylic acid concentration) in the pharmaceutical composition is than 120 mg/ml or less than 100 mg/ml or less than 75 mg/ml or less than 50 mg/ml or less than 30 mg/ml or less than 20 mg/ml and the weight ratio of lactoferrin and lysozyme to NSAID of from 10:1 to 1:10 (e.g., 8:1 to 1:8, 6:1 to 1:6, 2:1 to 1:2, 3:2 to 2:3).

The pharmaceutical compositions according to the disclosure may be in the form of a nasal spray, nasal drops, oral spray, oral rinse, or lozenge. The carrier of the pharmaceutical composition may be selected to provide residence time of the composition on the nasal and/or oral mucosa of at least 1 minute, or at least 5 minutes, or at least 10 minutes, or at least 15 minutes, or at least 20 minutes, or at least 25 minutes, or at least 30 minutes following application. In some embodiments, the composition for application to the nasal and/or oral mucosa comprises one or more antiviral and/or antimicrobial agents dispersed in a liquid carrier comprising from 1-99% (v/v) water or from 60-90% (v/v) water and from 10-40% (or from 20-30%) (v/v) of a polyol. In some embodiments, the pharmaceutically acceptable carrier is an aqueous solution comprising from 5-50% (v/v), or from 10-40% (v/v), or from 15-35% (v/v), or from 20-30% (v/v) 1,3-propanediol. The composition may be capable of being sprayed or ingested onto the mucosa, and is adapted to remain on the mucosa for at least 5 minutes (or at least 10 minutes, or at least 15 minutes, or at least 20 minutes, or at least 25 minutes, or at least 30 minutes) following application without substantially irritating or drying the mucosa.

The compositions may be administered by any suitable route, including orally, topically, nasally, and combinations thereof. In an embodiment, the composition is administered to nasal membranes. In an embodiment, the composition is administered to oral membranes. In an embodiment, the composition is administered using a device selected from the group consisting of an atomizer, an inhaler, a nebulizer, a spray bottle, and a spray pump. The composition may include a propellant or may be free of propellants.

The compounds and pharmaceutical compositions can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder, or they may achieve different effects (e.g., control of any adverse effects). In some embodiments, the pharmaceutical composition does not include a corticosteroid or less than 5% or less than 1% or less than 0.5% or less than 0.1% of a corticosteroid by weight of the composition. In various implementations, the pharmaceutical composition does not include flurbiprofen and/or cyclodextrin. In certain embodiments, the pharmaceutical composition comprises less than 1% flurbiprofen or less and/or less than 1% cyclodextrin. In some embodiments, the only NSAID in the pharmaceutical composition is one or more salicylate derivatives (e.g., acetylsalicylic acid). In some embodiments, the pharmaceutical composition comprises less than 5% or less than 1% or less than 0.5% or less than 0.1% of an NSAID other than salicylate derivatives (e.g., acetylsalicylic acid) by weight of the composition.

The pharmaceutical compositions may contain one or more additional components, for example, sweetening agents such as sucrose, fructose, lactose, aspartame or saccharin; flavoring agents such as peppermint, oil of wintergreen, or cherry; coloring agents; pH adjusting component, humectants, and preserving agents, to provide a pharmaceutically palatable preparation. Typical sweetening agents (sweeteners) useful in the composition include those that are both natural and artificial sweeteners. Sweetening agent used may be selected from a wide range of materials including water-soluble sweetening agents, water-soluble artificial sweetening agents, water-soluble sweetening agents derived from naturally occurring water-soluble sweetening agents, dipeptide based sweetening agents, and protein based sweetening agents, including mixtures thereof. Representative examples of moisturizing or humectant agents that are usable in the present invention include, without limitation, acetamide monoethanolamine urazole, aloe vera in any of its variety of forms (e.g., aloe vera gel, aloe vera extract, aloe vera concentrate), allantoin, guanidine, glycolic acid and glycolate salts (e.g., ammonium salt and quaternary alkyl ammonium salt), hyaluronic acid, lactamide monoethanolamine, polyethylene glycols, polyhydroxy alcohols (e.g., sorbitol, glycerol, hexanetriol, propylene glycol, butylene glycol, hexylene glycol and the like), sugars and starches, sugar and starch derivatives (e.g., alkoxylated glucose), and any combination thereof. Suitable flavoring agents include peppermint, oil, spearmint oil, wintergreen oil, clove, menthol, dihydroanethole, estragole, methyl salicylate, eucalyptol, cassia, 1-menthyl acetate, sage, eugenol, parsley oil, menthone, oxanone, alpha-irisone, alpha-ionone, anise, marjoram, lemon, orange, propenyl guaethol, cinnamon, vanillin, ethyl vanillin, thymol, linalool, limonene, isoamylacetate, benzaldehyde, ethylbutyrate, phenyl ethyl alcohol, sweet birch, cinnamic aldehyde, cinnamaldehyde glycerol acetal (known as CGA), and mixtures of the foregoing. Sweetening agents include sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame, cyclamate salts, and mixtures of the foregoing. In addition to the flavoring and sweetening agents, the compositions may include coolants, salivating agents, warming agents and numbing agents as optional ingredients. Coolants include carboxamides, menthol, paramenthan carboxamides, isopropylbutanamide, ketals, diols, 3-1-menthoxypropane-1,2-diol, menthone glycerol acetal, menthyl lactate, and mixtures thereof. Salivating agents include Jambu® (manufactured by Takasago). Warming agents include capsicum and nicotinate esters (such as benzyl nicotinate). Numbing agents include benzocaine, lidocaine, clove bud oil and ethanol. In some embodiments, the pharmaceutical composition may comprise one or more binders, such as, for example, carboxymethylcellulose, alginates, gelatin, polyvinyl pyrrolidone, sucrose and/or acacia.

The pharmaceutical composition may comprise one or more natural extracts and concentrates. Suitable whole leaf aloe vera concentrate may, for example, act as a carrying agent. The whole leaf aloe vera concentrate is present in an amount less than 10% (w/v) of the pharmaceutical composition, for example, from 2% (w/v) to 4% (w/v) or 0.1% (w/v) to 3% (w/v) or from 0.1% (w/v) to 2% (w/v) of the pain relieving composition. Although some studies may show that aloe extracts may confer anti-inflammatory properties, in some embodiments, the aloe is present in an amount less than is efficacious for such activity. Accordingly, the aloe may be considered part of the NSAID content or not part of the NSAID content, dependent on the concentration and dosage administered. In most embodiments, aloe extract is not considered part of the NSAID content. In some embodiments, the pharmaceutical composition comprises less than 10% (w/v) aloe.

In some embodiments, and particularly in oral sprays, the pharmaceutical composition is a solution such as an aqueous solution wherein a provided compound may be appropriately buffered by means of saline, acetate, phosphate, citrate, acetate or other buffering agents, which may be at any physiologically acceptable pH, generally from pH 4 to pH 7. Combinations of buffering agents may also be employed, such as phosphate buffered saline, a saline and acetate buffer, and the like. In the case of saline, a 0.9% saline solution may be employed. In the case of acetate, phosphate, citrate, acetate and the like, a 50 mM solution may be employed. In addition to buffering agents, suitable preservatives may be employed, to prevent or limit bacteria and other microbial growth. For example, the composition may have from 0.001% to 0.1% of a preservative by weight of the composition. One such preservative that may be employed is benzalkonium chloride (e.g., 0.05% (w/v) benzalkonium chloride).

In various embodiments, the pharmaceutical composition is administered orally, and more particularly, as an oral spray. A sweetener and flavor enhancers may also be included in the oral spray composition. Sweeteners may include fructose, dextrose, sucrose or the like. Non-artificial sweeteners may be included such as including fructose in an amount of from 8 to 15 weight percent of the oral spray composition (e.g. at 10 weight percent of the oral composition). One certain embodiment of the oral spray composition includes a flavor enhancer, such as peppermint, for example, in an amount of 0.5 to 2.0% (w/w) of the oral spray composition, including 1% (w/w) of the oral composition.

In accordance with another aspect of the present disclosure, a preservative may be added to the pharmaceutical composition to facilitate stability of the various ingredients. Any suitable preservative may be used in accordance with the present disclosure such as, for example, benzalkonium chloride, benzyl alcohol, and disodium EDTA. In some embodiments, the preservative includes a 50% solution of a preservative (e.g., benzalkonium chloride) admixed into the oral composition at a concentration of 0.01 to 0.02 percent by weight, for example 0.015 percent by weight.

In certain embodiments, the pharmaceutical composition may be formulated with the components as shown in Table 1 to achieve a therapeutic dose of NSAID within the therapeutic window described herein.

TABLE 1

| Component | Weight Percentage (w/v) | |
|---|---|---|
| Lactoferrin (e.g., apolactoferrin) | 0.01-0.1% | (e.g., 0.05%) |
| Lysozyme | 0.1-1% | (e.g., 0.5%) |
| Glycerol | 1%-10% | (e.g., 5%) |
| Sweetener | 10%-40% | (e.g., 30%) |
| Menthol | 0.1%-2% | (e.g., 1%) |
| Carboxymethyl Cellulose | 0.1%-2% | (e.g., 0.1%-1%, 1%) |
| Aloe Aqueous Extract | 0.1%-2% | (e.g., 0.1%-1%, 1%) |
| NSAID (e.g., Acetylsalicylic | 0.1%-1% | (e.g., 0.6%) |
| Acid (ASA)) | | |

In some embodiments, the pharmaceutical composition (e.g., the pharmaceutical composition according to Table 1) may be in the form of an oral spray composition. The oral spray composition may be used to deliver from 100 μL to 1 mL (e.g., from 300 μL to 700 μL, 500 μL) of spray composition per spray from an appropriate apparatus. In some embodiments, the pharmaceutical composition (e.g., the pharmaceutical composition according to Table 1) may be in the form of a more viscous or solid composition such as a gel or lozenge. In some embodiment, the lozenge may have the weight percentages indicated in Table 1. In certain embodiments, a lozenge may be formulated for a similar dosage of components as one or more (e.g. two, three, four) of the above described sprays. For example, the lozenge may be formed from removal of water from the liquid composition to form a syrup followed by solidification to form a lozenge. For example, a lozenge may have a total weight of each component as indicated in Table 2.

TABLE 2

| Component | Weight (mg) | |
|---|---|---|
| Lactoferrin (e.g., apolactoferrin) | 0.01-0.1 | (e.g., 0.05) |
| Lysozyme | 0.1-1 | (e.g., 0.5) |
| Glycerol | 1-10 | (e.g., 5) |
| Sweetener | 10-40 | (e.g., 30) |
| Menthol | 0.1-2 | (e.g., 1) |
| Carboxymethyl Cellulose | 0.1-2 | (e.g., 0.1-1, 1) |
| Aloe Aqueous Extract | 0.1-2 | (e.g., 0.1-1, 1) |
| NSAID (e.g., Acetylsalicylic Acid | 0.1-1 | (e.g., 0.6) |
| (ASA)) | | |

In an exemplary embodiment, the pharmaceutical composition is:
a) 0.05% (w/v) lactoferrin
b) 0.5% (w/v) lysozyme
c) 5% (w/v) glycerol
d) 30% (w/v) sweetener
e) 1% (w/v) menthol
f) 1% (w/v) carboxymethyl cellulose
g) 1% (w/v) aloe aqueous extract; and
h) 0.6% (w/v) acetylsalicylic acid.
   in an aqueous buffered carrier. In certain embodiments, the pharmaceutical composition (e.g., as disclosed in Table 1 or Table 2, or in the exemplary embodiments disclosed herein) including the NSAIDs (e.g., salicylate and derivatives thereof including salicylic acid and acetylsalicylic acid) may increase the anti-inflammatory response as compared to the combined results of an otherwise identical composition without the NSAIDs and the NSAIDs alone (e.g., in aqueous buffer). For example, the NSAIDs in combination with the lactoferrin and/or lysozyme may provide decreased prostaglandin (e.g., PGE2) production and/or decreased interleukin-8 (IL-8) production as compared to the combined result of the NSAIDs alone and an otherwise identical composition without the NSAIDs.

In a further embodiment, the present disclosure relates to kit comprising a stable fixed dose, aqueous pharmaceutical composition of the present disclosure contained in a container for nasal and/or oral administration and a package insert containing instructions about the use of the pharmaceutical composition. In one embodiment, the container is part of a sprayer which has an actuator. When the actuator is actuated, the composition is delivered in the form of a spray. In a further embodiment, the pharmaceutical composition is contained in a sprayer, and has, on deliver a spray of the composition to a human nose, a spray pattern having a longest axis of 15-75 mm, a shortest axis of 10-65 mm, and an ellipticity of 1-2. In the context of present disclosure, the pharmaceutical composition when delivered as a nasal and/or oral spray using a sprayer yields a specific spray pattern and spray droplet size. The spray pattern can be determined by various known techniques such as with an axisymmetric drop shape analysis (ADSA) with Nasal Spray Products Universal Actuator (NSP UA) set up (Innova System) and the spray droplet size distribution can be determined by various known techniques such as with a Malvern Spraytec with NSPUA set up (Innova System). The following describes a typical procedure for characterizing droplet size distribution of the spray—The sprayer is loaded with a composition as described above and primed by an actuating pump via an actuator until a fine mist appears out of the nozzle of the sprayer. A commercially available laser diffraction instrument is arranged so that the nozzle is 3 cm or 6 cm below the laser beam of the laser diffraction instrument. The pump is actuated with a conventional mechanical actuator using a constant force. The resulting spray of the composition crosses the laser beam. Data are collected for D₁₀, D₅₀, D₉₀, SPAN, and % Volume <10 μm. The average values for each of these parameters for three sprays are calculated. The aqueous suspension or aqueous solution can be administered as a drop or any other form suitable for topical administration.

In some embodiments, the aqueous suspension is provided in the form of an oral spray or nasal spray wherein the suspension is administered in a single unit-dose container or multi-dose container. Suitable single unit-dose containers or multi-dose containers include, but are not limited to, glass, aluminum, polypropylene or high-density polyethylene, for example, high density polyethylene containers produced using a blow-fill-seal manufacturing technique.

In certain additional embodiments, the disclosure provides a multi dosage composition, comprising: (a) a multi-unit dosage of a pharmaceutical composition of the present disclosure; and (b) a container comprising: (i) a squeezable chamber holding the multi dosage of the composition and having an opening wherein the dosage exits the opening when the squeezable chamber is squeezed; and (ii) a closure mechanism removably attached to the opening of the squeezable chamber. In certain embodiments, the multi dosage container is made of a moldable polymer. In such embodiments, suitable polymers include, but are not limited to, polyethylene, polypropylene (PP), polystyrene (PS), nylon (Ny), polyvinyl chloride (PVC), polyethylene terephthalate (PET), polycarbonate (PC), polyoxymethylene (POM), polysulfone (PSF), polyethersulfone (PES), polyacrylate (PAR), and polyamide (PA). In certain embodiments, polymers include polyethylene, particularly medium-density polyethylene (MDPE) (or branched polyethylene) or high-density polyethylene (HDPE) (or linear, polyethylene). In one embodiment, the multi dose container is made of high density polyethylene (HDPE).

The composition of the present disclosure may be delivered to the oral cavity through the mouth by way of a fine spray mist. The method includes the steps of obtaining an oral composition in accordance with the present disclosure for delivery into the oral cavity. The method further includes the step of applying the oral composition to the oral cavity with a spray applicator. Practitioners will appreciate that any suitable applicator may be used. For example, the applicator may be configured to hold from 100-150 metered doses of the composition, wherein the metered dose is from 0.1 ml to 1 ml (e.g., from 0.25 to 0.75 ml including 0.5 ml).

Other means for delivering the nasal and/or oral spray, such as inhalation via a metered dose inhaler (MDI), may also be used. Several types of MDIs are regularly used for administration by inhalation. These types of devices can include breath-actuated MDIs, spacer/holding chambers in combination with MDIs, and nebulizers. Metered dose inhalers are an inhalation delivery system comprising, for example, a canister containing a mixture of an active agent and a propellant optionally with one or more excipients, a metered dose valve, an actuator, and a mouthpiece. The canister is may be filled with a suspension of an active agent, such as an oral spray composition as described herein, and a propellant, such as one or more hydrofluoroalkanes [e.g. 1,1,1,2-tetrafluoroethane (HFA-134a) and 1,1,1,2,3,3,3-heptafluoropropane (HFA-227)], chlorofluorocarbons, and alcohols such as ethanol, isopropanol, butanol, propanol or mixtures thereof. However, typically, the composition is free of propellants. When the actuator is depressed a metered dose of the suspension is aerosolized for inhalation. Particles comprising the active agent are propelled towards the mouthpiece where they may then be inhaled by a subject.

The pharmaceutical product may comprise:
(a) a body configured to be inserted into an oral passage for dispensing an oral spray composition as disclosed herein;
(b) a reservoir in fluid communication with the orifice, wherein the oral spray composition is contained in the reservoir;
(c) a pump mechanism capable of expelling the oral spray composition through the orifice in appropriate sized aerosolized droplets; capable of coating the oral mucosa (e.g. mucosa of the throat) of a user.
   In some embodiments, a single actuation of the pump mechanism is configured to expel from 100 μL to 1000 μL (e.g., 200 μL to 800 μL, 400 μL to 600 μL, 500 μL) of the oral spray composition. For example, each spray may expel less than 75 mg or less the 60 mg or less than 50 mg or less than 25 mg or less than 15 mg or less than 10 mg of NSAIDs (e.g., acetylsalicylic acid). In certain embodiments, the oral spray composition comprises:
a) from 1 to 10 mg/ml of the one or more NSAIDs;
b) optionally from 0.1 to 1 mg/ml of lactoferrin;
c) optionally from 1 to 10 mg/ml of lysozyme;
c) optionally from 10 to 100 mg/ml of glycerol;
e) optionally from 100 to 400 mg/ml of sweetener;
f) optionally from 1 to 20 mg/ml menthol;
g) optionally from 1 to 20 mg/ml carboxymethyl cellulose; and
h) optionally from 1 to 20 mg/ml aloe.

The composition may be delivered to an individual in any suitable dosage. In accordance with one embodiment of the disclosure, the oral spray applicator is configured to supply a unit dose of from 0.1 ml to 1 ml (e.g., from 0.25 to 0.75 ml including 0.5 ml) of composition to the individual each time a pump associated with the spray applicator is activated (e.g., 0.5 ml/spray). In certain embodiments, the composition is delivered by applying 2 sprays in the mouth within 10 to 30 minutes.

Medicaments for the treatment or prophylaxis of inflammatory conditions of the mucosa such as sore throat are provided herein. Typically, the medicament comprises less than 75 mg of one or more NSAIDs (e.g., acetylsalicylic acid). In some embodiments, the medicaments is the pharmaceutical composition described herein. In various implementations, acetylsalicylic acid for the use in the preparation of medicament is disclosed, wherein the medicament comprises less than 75 mg acetylsalicylic acid (e.g., less than 50 mg acetylsalicylic acid, less than 25 mg acetylsalicylic acid, less than 20 mg acetylsalicylic acid, less than 15 mg salicylic acid). In certain embodiments, acetylsalicylic acid for the use in the preparation of medicament is disclosed, wherein the medicament comprises less than 75 mg/ml acetylsalicylic acid (e.g., less than 50 mg/ml acetylsalicylic acid, less than 25 mg/ml acetylsalicylic acid, less than 20 mg/ml acetylsalicylic acid, less than 15 mg/ml acetylsalicylic acid).

Methods for the treatment or prophylaxis of sore throat in a subject in need thereof are provided comprising:
a) inserting the portion of the pharmaceutical product comprising the oral spray configured to be inserted into an oral passage into the oral passage of the subject; and
b) actuating the pump mechanism to administer the oral spray composition to the subject.
   In some embodiments, the inserting and actuating steps are repeated at a time point more than 10 minutes after the previous of the administration oral spray composition.

Methods for the treatment or prophylaxis of sore throat in a subject in need thereof are also disclosed here, wherein the method comprises administration of these pharmaceutical composition to the subject in need thereof. In some embodiments, the method for the treatment or prophylaxis of sore throat in a subject in need thereof may comprise administering less than 75 mg (e.g., less than 50 mg, less than 25 mg, less than 15 mg) of a plurality of NSAIDs to the throat. The dosage regimens described herein allow for anti-inflammatory responses to be produced on the mucosa of the user thereby treating and/or preventing the anti-inflammatory response that would have been present without such administration. For example, less than 325 mg (e.g., less than 100 mg, less than 75 mg, less than 50 mg, less than 25 mg, less than 20 mg, less than 15 mg) of the plurality of NSAIDs are administered in 24 hours. In some embodiments, less than 325 mg (e.g., less than 100 mg, less than 75 mg, less than 50 mg, less than 25 mg, less than 20 mg, less than 15 mg) of the plurality of NSAIDs are administered in 12 hours. In some embodiments, the plurality of NSAIDs are administered within a time period of less than one hour or less than 30 minutes. In some embodiments, the plurality of NSAIDs are administered daily within a time period of less than one hour or less than 30 minutes. In certain embodiments, the sore throat is associated with inflammation resulting in increased prostaglandin production. In some embodiments, the methods for the treatment or prophylaxis of sore throat may have minimal (e.g., within 20% or within 10% or within 5%) or no effect on barrier integrity of the mucosa (e.g., as measured by TEER).

### EXAMPLES

The following examples illustrate specific aspects of the instant description. The examples should not be construed as limiting, as the example merely provides specific understanding and practice of the embodiments and its various aspects.

### Example 1

Various compositions were tested for their ability to protect a 3D model of human airway epithelium, constituted with primary human epithelial cells freshly isolated from nasal (MucilAir™ nasal pool), or tracheal or bronchial biopsies (MatTek PE-200-6.5). MucilAir™ and MatTek tissues are reconstituted human 3D tissue from airways and lung surgical pieces, fully differentiated, pseudostratified in vitro epithelium. MucilAir™_Pool is of nasal polyp origin and reconstituted with a mixture of cells isolated from 14 different donors. Cultured at the air-liquid interface, the model displays high trans-epithelial electrical resistance, cilia beating as well as mucus production, demonstrating the full functionality of the epithelial tissue. MatTek tissues are from tracheo-bronchial origin. The respiratory epithelium lining the nose and throat have very similar histology and architecture. These two different models reconfirm that models from different origin have similar responses to treatment in the case that one model may have more underlying inflammation, or other variability with respect to its retrieval than another.

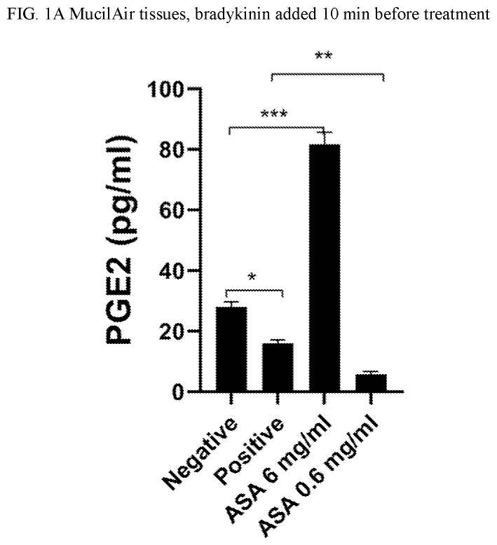

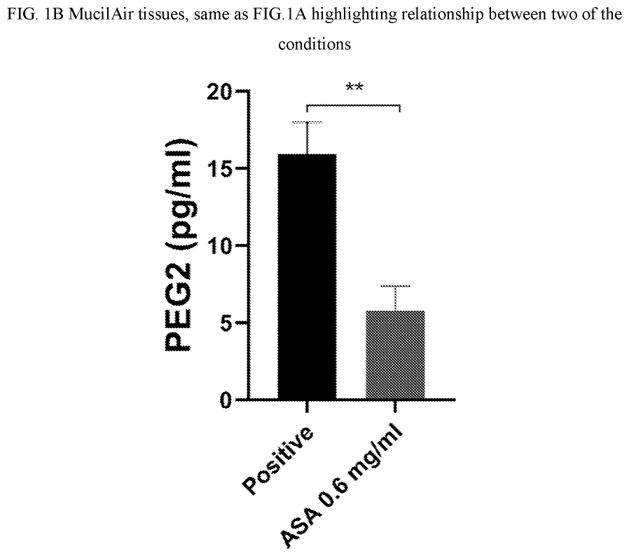

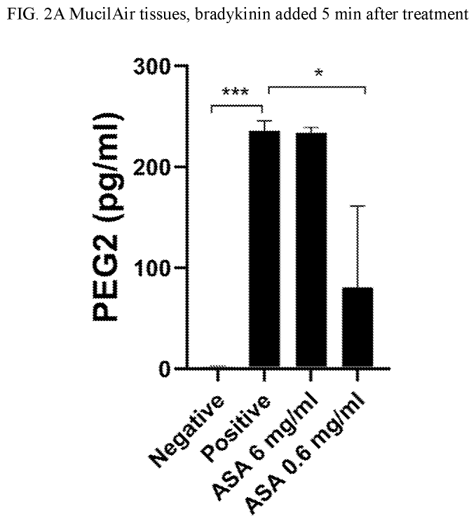

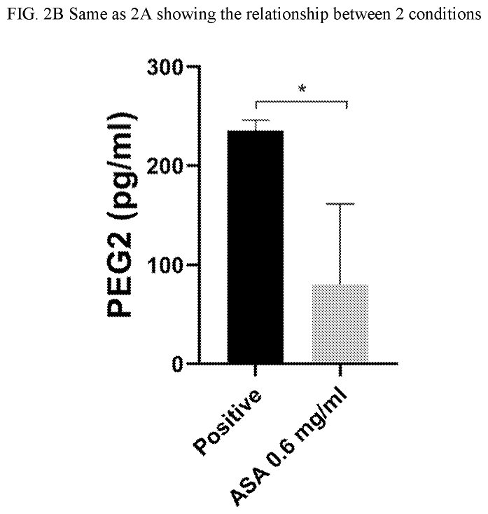

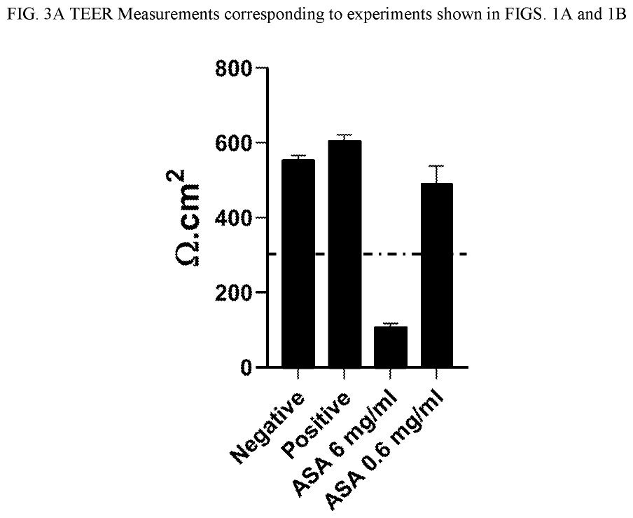

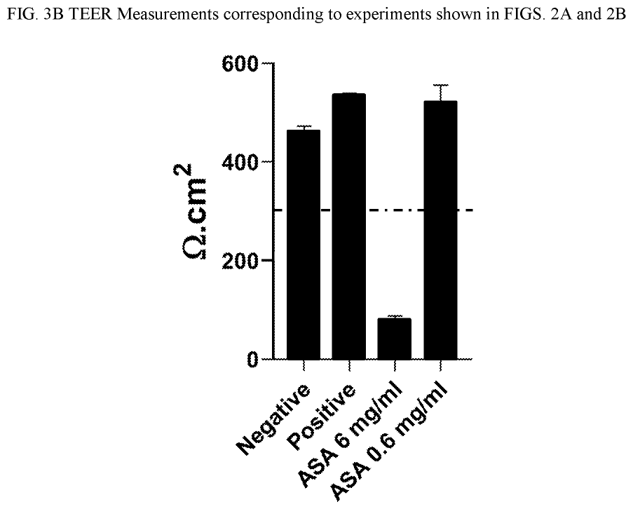

Compositions were formulated with various concentrations of acetylsalicylic acid (ASA 0.6 mg/ml or ASA 6 mg/ml) in an aqueous saline solution vehicle such as 0.9% NaCl, 1.25 mM CaCl₂, 10 mM HEPES. 10 μL of each formulation was added apically either to the MucilAir™ or MatTek tissues. Formulations were either added 10 minutes after activation of an inflammatory response in the media (FIGS. 1A-B, 3A, 4A), or 5 minutes before activation of an inflammatory response in the media (FIGS. 2A-B, 3B, 4B). Due to the rate of saliva production and concomitant dilution of acetylsalicylic acid on the mucosa, administration of each concentration in vitro correlates to a 10× concentration in product form. For example, 0.6 mg/ml ASA in the in vitro measurements corresponds administration of 6 mg/ml ASA in compositions, and 6 mg/ml in vivo corresponds to administration of 60 mg/ml in compositions.

Typically, the inflammatory cytokine bradykinin stimulates the prostaglandin pathway (through arachidonic acid) which then stimulates IL-8, another inflammatory cytokine. As can be seen, acetylsalicylic acid downregulates prostaglandin by inhibiting COX-2 (which stimulates prostaglandin) and also by inhibiting NFkB which leads to transcription of IL-8. The inflammatory response of the prostaglandin pathway was stimulated through the addition of 10 μL of 25 mg/ml bradykinin protein was applied to the apical side of the MucilAir™ media.

Four hours following bradykinin application various measurements of the inflammatory response were measured. Epithelia were washed twice with MucilAir™ culture medium in order to clean the inoculum. Liquid aliquots from the basal medium were collected and stored at −80° C. for future study.

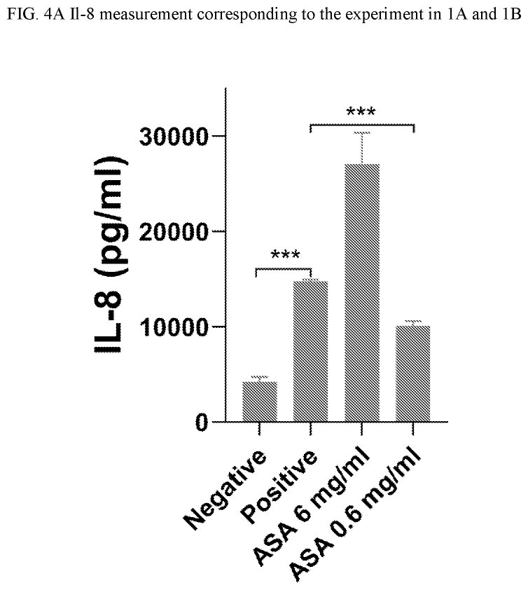

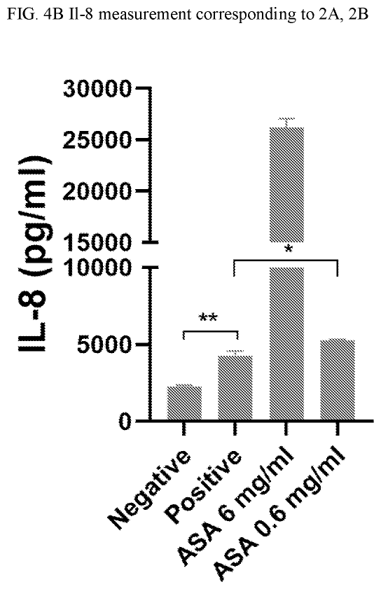

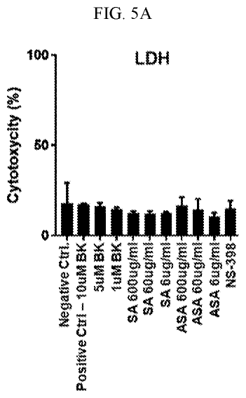

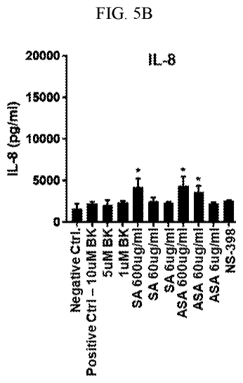

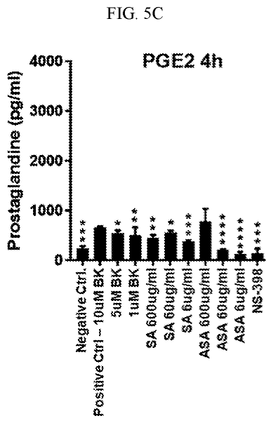

Prostaglandin E2 or IL-8 enzyme-linked immunosorbent assays (ELISA) were performed on the liquid aliquots from the basal medium to determine the amount of prostaglandin E2 produced as a result of inflammatory conditions initiated in each test condition. FIG. 1A shows the resultant measured prostaglandin concentration measured on aliquots taken from media without bradykinin application (negative), with bradykinin application (positive control), with 6 mg/ml ASA, and 0.6 mg/ml ASA. As can be seen, 6 mg/ml (corresponding to compositions comprising 60 mg/ml) increased the inflammatory response in a statistically significant matter. Although, acetylsalicylic acid is typically administered in doses greater than these measured, this dosage increased the inflammatory response. However, as shown in FIGS. 1A and 1B, 0.6 mg/ml administration resulted in an anti-inflammatory response to bradykinin administration. When the formulations were administered prior to bradykinin application, application of 6 mg/ml ASA resulted in no statistical significance with respect to positive control (see FIG. 2A). However, the lower dose of 0.6 mg/ml did inhibit inflammation in a statistically significant manner (see FIGS. 2A and 2B). Moreover, IL-8 concentrations were significantly greater in both therapeutic (FIG. 4A and prophylactic (FIG. 4B) modalities in the 6 mg/ml ASA administration. A similar result was not observed in the 0.6 mg/ml measurements.

Tissue integrity was also monitored using transepithelial electrical resistance (“TEER”) measurements. TEER is a dynamic parameter that reflects the state of epithelia and the barrier function which can be affected by several factors. For example, if holes were present or if cellular junction were broken, the TEER values would be generally below 300 Ω cm². In contrast, when epithelia are not damaged, the TEER values are typically above 300 Ω cm². A notable decrease of the TEER values (but >300 Ω cm²) generally reflects an activation of the ion channels.

For TEER measurements, 200 μL of warm MucilAir™ media or MatTek TEER buffer was applied on the apical side of each insert. A Millicell ERS-2 Voltohmmeter (#MERS00002 Millipore) with dual electrodes was washed with 70% ethanol and saline solution (0.9% NaCl, 1.25 mM CaCl₂, 10 mM HEPES). The long stem of the electrode is inserted through the gap of the MucilAir™ insert and was supported on the bottom of the well while the short stem was suspended in the apical media. The resistance (Ω) is read on the Voltohmmeter and the TEER value was calculated with the following formula: TEER (Ω·cm²)=(resistance value (Ω)−100 (Ω))×0.33 (cm²). TEER measurements were taken at 48 hours PI.

The resistance measurements from on the epithelia for treatments are shown in FIGS. 3A and 3B. As can be seen, 6 mg/ml acetylsalicylic acid administration resulted in dramatic decreases in barrier function of the epithelia below the 100 Ω·cm² threshold. However, a similar decrease in barrier function is not shown for administration of 0.6 mg/ml ASA. These data demonstrate the existence of a therapeutic window on the mucosa, below that which is typically administered, where acetylsalicylic acid is effective at providing an anti-inflammatory response to inflammation with no effect on barrier function. Furthermore, above this window (yet still below typical administration doses), administration of acetylsalicylic acid increases inflammation and degrades barrier function. As can be seen, this NSAID dosage range is able to block the prostaglandin cytokine pathway (as measured by decreased prostaglandin production) thereby preventing or treating inflammatory conditions of the mucosa. The bradykinin induced increase of PGE-2 can be inhibited by acetylsalicylic acid (aspirin), a non-selective Cox inhibitor, as expected but in a dose dependent manner. Acetyl-salicylic acid did not appreciably decrease levels of IL-8 protein, despite significantly decreasing PGE-2 levels.

### Example 2

Apart from the COX pathway, arachidonic acid (AA) is also responsible for the generation of leukotrienes via lipoxygenase. Without wishing to be bound by theory, inhibiting the COX pathway with COX inhibitors may result in the shunting of AA to the lipoxygenase pathway resulting in the production of more leukotrienes. One of the major lipoxygenase products, LTB4, has been shown to stimulate synthesis and release IL-8.

A549 cells and in the two ex-vivo models were studied, illustrating a small therapeutic window in which NSAIDs inhibit PGE-2 and IL-8 release. At a ten-fold higher concentration than this therapeutic dose, IL-8 increased to levels 2-5 times higher than with bradykinin stimulation alone. IL-8 is known to be an inflammatory cytokine, and at high levels of protein expression, we observed negative cytopathic effect such as decrease in TEER and increase in LDH.

A549 cells were seeded at a density of 6,400 cells per well in complete growth media. After a day, the growth media was replaced with serum-free media. On day 2, the media was removed and replaced with 180 μL of various test compositions illustrated in FIGS. 5A-C (600 g/ml sialic acid (SA), 60 μg/ml SA, 6 μg/ml SA, 600 μg/ml acetylsalicylic acid (ASA), 60 g/ml ASA, 6 μg/ml ASA, a composition comprising the COX-2 specific inhibitor NS-398). Following 30 minutes of incubation with each test composition, the cell mixtures were challenged with 20 μL various concentrations of lyophilized bradykinin (BK) which was reconstituted the day of application (positive control for each experiment was the 10 M BK challenge).

Lactate dehydrogenase (LDH) is a stable cytoplasmic enzyme that is rapidly released into the culture medium upon rupture of the plasma membrane. Samples were taken to measure the LDH in each test condition. The amount of the released LDH was quantified using the absorbance of each sample at 490 nm with a microplate reader. The high control value was obtained by 10% Triton X-100 treatment 24 hours prior to the assay and corresponds to 100% cytotoxicity. FIG. 5A shows the measured cytoxicity of these cells following BK challenge and administration of the indicated components. Additionally, ELISA assays were measured to quantify the PGE-2 and IL-8 cytokine production as well. The statistical significance of the data was measured by t tests and is represented by “*” (P<0.05) with decreasing p value with increased “*” between samples and positive control. These illustrate prevention of PGE-2 release by the different compounds while IL-8 does not follow a similar pattern.

### Example 3

Since COX-2 is the major Cox expressed in respiratory epithelial cells, the negative cytopathic effect of NSAID administration in these therapeutic windows may not be due to an imbalance in Cox enzymes. Instead, by inhibiting COX-2, arachidonic acid may be shunted to producing leukotrienes.

Given bradykinin's important role in the inflammatory response and pain signaling, it is important for the signaling pathways associated therewith to be tightly regulated. Ex-vivo models were prepared illustrating that bradykinin's stimulation of PGE2 and IL-8, reaches a saturation point similar to Example 1.

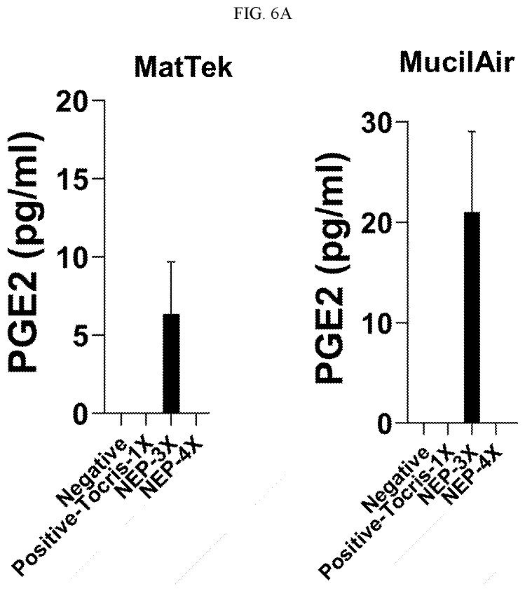

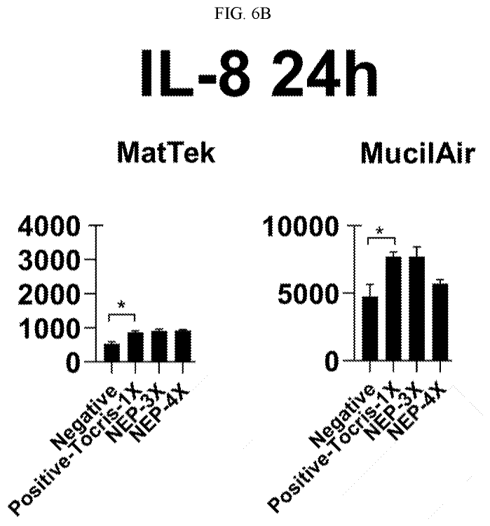

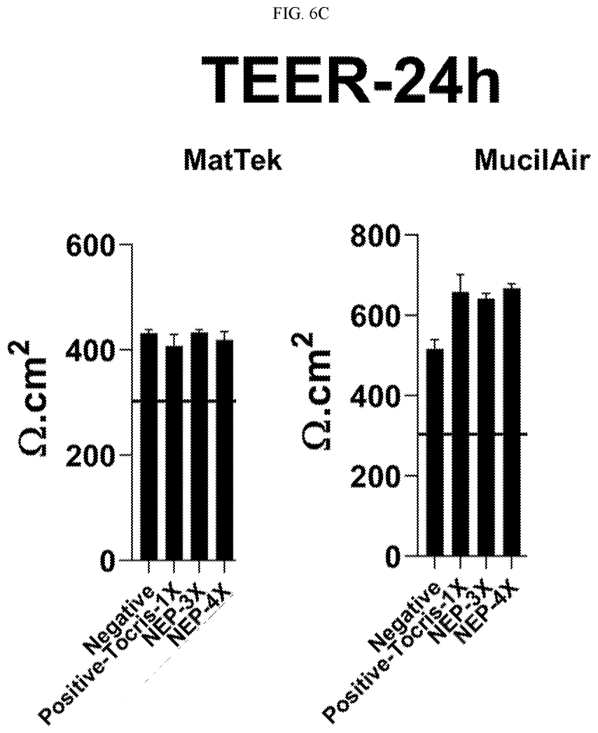

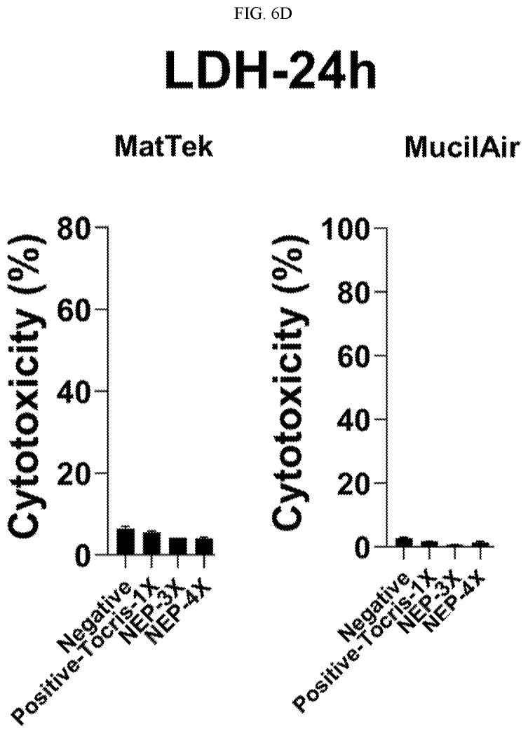

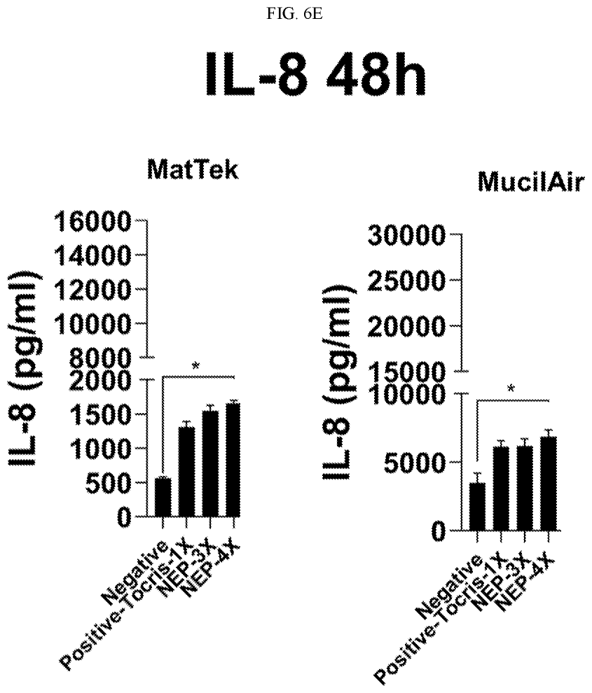

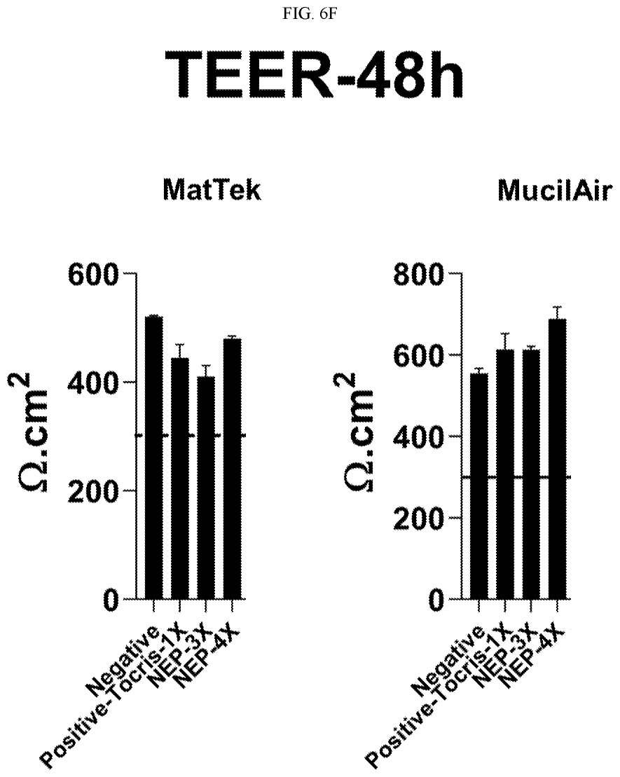

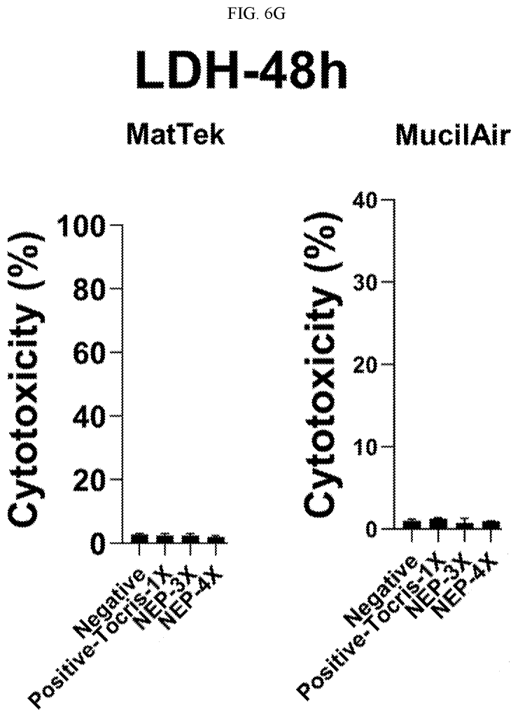

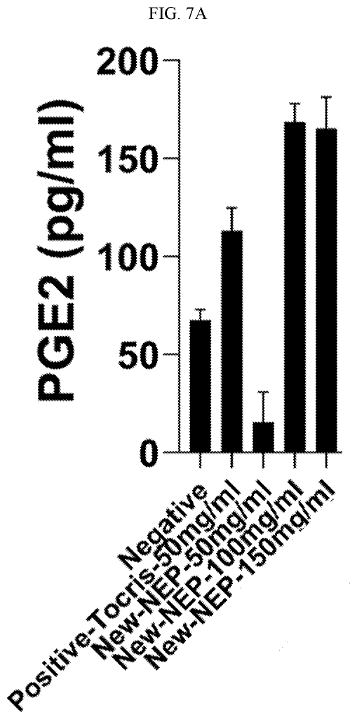

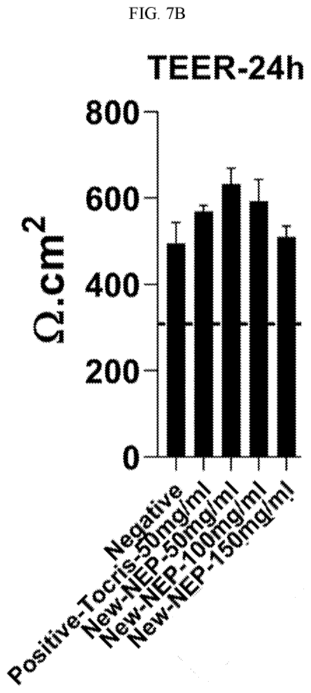

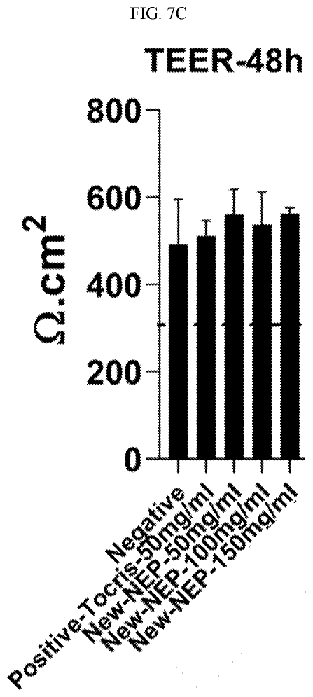

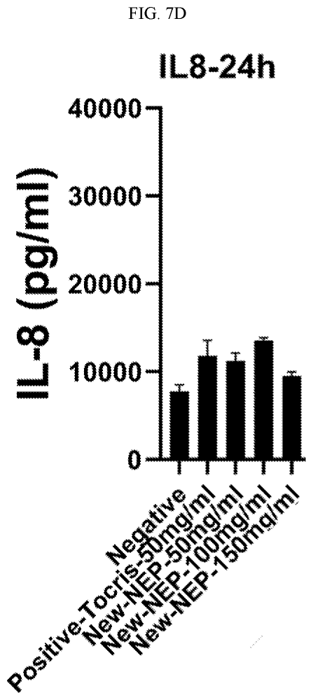

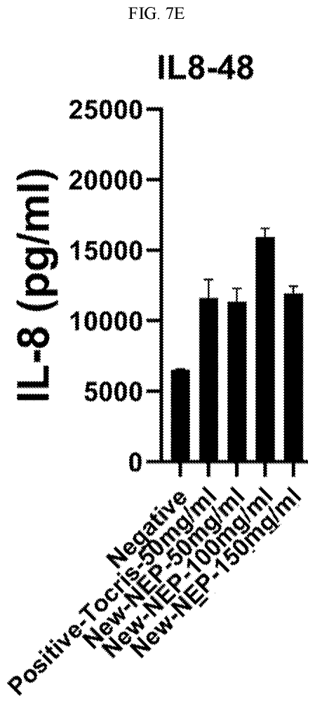

MatTek (tracheal bronchial tissue) or MucilAir (nasal polyps) 3D inserts were placed in pre-warmed media corresponding to each cell system and incubated for 15 minutes at 37° C. L of saliva (negative control) or treatment was added to the apical surfaces. 5 minutes later, 10 μL of saliva or bradykinin was added to the apical side as well. Bradykinin was obtained from Tocris or New England Peptide (NEP) as indicated. At 4 hours, samples from the basal media were collected and frozen at −80° for PGE-2 ELISA measurements. At 24 hours, the inserts were moved to fresh basal media (corresponding to each cell system) and 200 L of media were added to each apical side for TEER analysis as described previously. The plate was then incubated for 5 minutes. Following TEER measurements, the apical solution was removed and 10 μL of each treatment (or saliva) was added to the apical surface. No bradykinin was added. TEER was measured again at 48 hours. Additionally, at 24 hours and 48 hours, basal media was collected and frozen for IL-8 and LDH analyses. FIGS. 6A-G show the results of these measurements at different concentrations and sources of bradykinin (BK 1X=16 mg/ml for MatTek and 50 mg/ml for MucilAir) in tracheal bronchial tissue (MatTek) and nasal polyp (MucilAir) systems. A similar set of experiments was performed on MucilAir media at different bradykinin concentrations (FIGS. 7A-7E).

At the maximum concentration of PGE-2 and IL-8, TEER and LDH levels remained in the normal range, suggesting that there was no cytotoxicity. However, as noted earlier, a dose of acetyl salicylic acid above the therapeutic window increased PGE2 and IL-8 above these levels. Therefore, another pathway might be involved in the therapeutic windows for NSAIDs described herein. As seen by the TEER and LDH measurements, the higher levels of PGE2 and IL-8 induced by this pathway may not be healthy for the respiratory epithelium, Receptor saturation or downstream saturation or regulation might be responsible for controlling PGE2 and IL-8 levels under physiological conditions. Alternatively, the two pathways might represent different levels or tiers of the inflammatory response, one which might lead to excess inflammation analogous to a “cytokine storm.” Nevertheless, such mechanisms may be operative and/or leveraged in the lower therapeutic NSAID doses of the present disclosure.

### SPECIFIC EMBODIMENTS

Non-limiting specific embodiments are described below each of which is considered to be within the present disclosure.

Specific Embodiment 1. A pharmaceutical composition comprising one or more pharmaceutically acceptable excipients, carriers, and/or diluents and one or more non-steroidal anti-inflammatory drugs (NSAID), wherein the total concentration of NSAIDs is less than 75 mg/ml.

Specific Embodiment 2. The pharmaceutical composition according to Specific Embodiment 1, wherein the NSAIDs comprise acetylsalicylic acid (ASA).

Specific Embodiment 3. The pharmaceutical composition according to Specific Embodiment 2, wherein the concentration of the acetylsalicylic acid is less than 10 mg/ml.

Specific Embodiment 4. The pharmaceutical composition according to any one of Specific Embodiments 1-3, wherein the total concentration of NSAIDs is less than 20 mg/ml.

Specific Embodiment 5. The pharmaceutical composition according to any one of Specific Embodiments 1-4, wherein the total NSAID content of the composition is more than 90% or more than 95% or more than 99% acetylsalicylic acid by weight of the NSAID content.

Specific Embodiment 6. The pharmaceutical composition according to any one of Specific Embodiments 1-4, wherein acetylsalicylic acid is the only NSAID in the composition.

Specific Embodiment 7. The pharmaceutical composition according to any one of Specific Embodiments 1-4, wherein acetylsalicylic acid and aloe extract are the only NSAIDs in the composition.

Specific Embodiment 8. The pharmaceutical composition according to any one of Specific Embodiments 1-7, further comprising lactoferrin and lysozyme.

Specific Embodiment 9. The pharmaceutical composition according to Specific Embodiment 8, wherein the weight ratio of the lactoferrin to lysozyme is 1:1 to 1:100.

Specific Embodiment 10. The pharmaceutical composition according to Specific Embodiment 8 or 9, wherein the weight ratio of lactoferrin and lysozyme to NSAID is from 10:1 to 1:10.

Specific Embodiment 11. The pharmaceutical composition according to any one of Specific Embodiments 1-10, wherein the pharmaceutical composition does not comprise a corticosteroid.

Specific Embodiment 12. The pharmaceutical composition according to any one of Specific Embodiments 1-11, wherein the pharmaceutical composition comprises:
a) from 1 to 10 mg/ml of the one or more NSAIDs;
b) from 0.1 to 1 mg/ml of lactoferrin;
c) from 1 to 10 mg/ml of lysozyme;
c) from 10 to 100 mg/ml of glycerol;
e) from 100 to 400 mg/ml of sweetener;
f) from 1 to 20 mg/ml menthol;
g) from 1 to 20 mg/ml carboxymethyl cellulose; and
h) from 1 to 20 mg/ml aloe.

Specific Embodiment 13. The pharmaceutical composition according to any one of Specific Embodiments 1-12, wherein the pharmaceutical composition is formulated as a lozenge.

Specific Embodiment 14. The pharmaceutical composition according to Specific Embodiment 13, wherein the lozenge comprises from 1 to 10 mg of the one or more NSAIDs.

Specific Embodiment 15. The pharmaceutical composition according to Specific Embodiment 13, wherein the lozenge comprises from 1 to 10 mg of acetylsalicylic acid.

Specific Embodiment 13. The pharmaceutical composition according to Specific Embodiment 13, wherein said lozenge comprises from 0.1 to 10 mg of acetylsalicylic acid and more than 90% (e.g., more than 95%, more than 99%, more than 99.9%) of the one or more NSAIDs is acetylsalicylic acid by weight of the NSAIDs.

Specific Embodiment 16. The pharmaceutical composition according to any one of Specific Embodiments 13-22, wherein the lozenge comprises:
a) from 1 to 10 mg of the one or more NSAIDs;
b) from 0.1 to 1 mg of lactoferrin;
c) from 1 to 10 mg of lysozyme;
c) from 10 to 100 mg of glycerol;
e) from 100 to 400 mg of sweetener;
f) from 1 to 20 mg menthol;
g) from 1 to 20 mg carboxymethyl cellulose; and
h) from 1 to 20 mg aloe.

Specific Embodiment 17. The pharmaceutical composition according to any one of Specific Embodiments 1-12, wherein the pharmaceutical composition is formulated as an oral spray.

Specific Embodiment 18. A method for the treatment or prophylaxis of sore throat in a subject in need thereof comprising administration of the pharmaceutical composition according to any one of Specific Embodiments 1-17.

Specific Embodiment 19. The method according to Specific Embodiment 18, wherein less than 325 mg (e.g., less than 100 mg, less than 75 mg, less than 50 mg, less than 25 mg, less than 20 mg, less than 15 mg) of the plurality of NSAIDs are administered in 24 hours.

Specific Embodiment 20. The method according to Specific Embodiment 18, wherein less than 325 mg (e.g., less than 100 mg, less than 75 mg, less than 50 mg, less than 25 mg, less than 20 mg, less than 15 mg) of the plurality of NSAIDs are administered in 12 hours.

Specific Embodiment 21. The method according to any one of Specific Embodiments 18-20, wherein the sore throat is caused by inflammation resulting in increased prostaglandin production.

Specific Embodiment 22. A pharmaceutical product comprising:
(a) a body configured to be inserted into an oral passage for dispensing the oral spray composition according to Specific Embodiment 17;
(b) a reservoir in fluid communication with the orifice, wherein the oral spray composition is contained in the reservoir;
(c) a pump mechanism capable of expelling the oral spray composition through the orifice in appropriate sized aerosolized droplets; capable of coating the oral mucosa (e.g. mucosa of the throat) of a user.

Specific Embodiment 23. The pharmaceutical product according to Specific Embodiment 22, wherein the pump mechanism is configured to expel from 100 μL to 1000 μL of the oral spray composition.

Specific Embodiment 24. A method for the treatment or prophylaxis of sore throat in a subject in need thereof comprising:

a) inserting the portion of the pharmaceutical product according Specific Embodiment 22 or 23 configured to be inserted into an oral passage into the oral passage of the subject; and

b) actuating the pump mechanism to administer the oral spray composition to the subject.

Specific Embodiment 25. The method according to Specific Embodiment 24, wherein the inserting and actuating steps are repeated at a time point more than 10 minutes after the previous of the administration oral spray composition.

Specific Embodiment 26. A method for the treatment or prophylaxis of sore throat comprising

Specific Embodiment 27. A method for the treatment of sore throat in a subject in need thereof comprising administering less than 75 mg (e.g., less than 50 mg, less than 25 mg, less than 15 mg) of a plurality of NSAIDs to the throat.

Specific Embodiment 28. The method according to Specific Embodiment 27, wherein more than 90% of the plurality of NSAIDs is acetylsalicylic acid by weight of the plurality of NSAIDs.

Specific Embodiment 29. The method according to Specific Embodiment 27 or 28, wherein less than 325 mg (e.g., less than 100 mg, less than 75 mg, less than 50 mg, less than 25 mg, less than 20 mg, less than 15 mg) of the plurality of NSAIDs are administered in 24 hours.

Specific Embodiment 30. The method according to Specific Embodiment 27 or 28, wherein less than 325 mg (e.g., less than 100 mg, less than 75 mg, less than 50 mg, less than 25 mg, less than 20 mg, less than 15 mg) of the plurality of NSAIDs are administered in 12 hours.

Specific Embodiment 31. The method according to any one of Specific Embodiments 19-22, and 25-30, wherein the administration has minimal or no effect on the barrier integrity of the mucosa (e.g., as measured by TEER) as compared to an otherwise identical mucosa that had not been administered the one or more NSAIDs.

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications and variances which fall within the scope of the appended claims.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

1. A pharmaceutical composition comprising one or more pharmaceutically acceptable excipients, carriers, and/or diluents and one or more non-steroidal anti-inflammatory drugs (NSAID), wherein the total concentration of NSAIDs is less than 75 mg/ml.

2. The pharmaceutical composition according to claim 1, wherein said NSAIDs comprise acetylsalicylic acid (ASA).

3. The pharmaceutical composition according to claim 2, wherein the concentration of said acetylsalicylic acid is less than 10 mg/ml.

4. The pharmaceutical composition according to claim 1, wherein said total concentration of NSAIDs is less than 20 mg/ml.

5. The pharmaceutical composition according to claim 1, wherein the total NSAID content of said composition is more than 90% or more than 95% or more than 99% salicylate and derivatives thereof by weight of the NSAID content.

6-7. (canceled)

8. The pharmaceutical composition according to claim 1, further comprising lactoferrin and lysozyme.

9. The pharmaceutical composition according to claim 8, wherein the weight ratio of said lactoferrin to lysozyme is 1:1 to 1:100.

10. The pharmaceutical composition according to claim 8, wherein the weight ratio of lactoferrin and lysozyme to NSAID is from 10:1 to 1:10.

11. (canceled)

12. The pharmaceutical composition according to claim 1, wherein said pharmaceutical composition comprises:
a) from 1 to 10 mg/ml of said one or more NSAIDs;
b) from 0.1 to 1 mg/ml of lactoferrin;
c) from 1 to 10 mg/ml of lysozyme;
c) from 10 to 100 mg/ml of glycerol;
e) from 100 to 400 mg/ml of sweetener;
f) from 1 to 20 mg/ml menthol;
g) from 1 to 20 mg/ml carboxymethyl cellulose; and
h) from 1 to 20 mg/ml aloe.

13. The pharmaceutical composition according to claim 1, wherein said pharmaceutical composition is formulated as a lozenge.

14. (canceled)

15. The pharmaceutical composition according to claim 13, wherein said lozenge comprises from 0.1 to 10 mg of acetylsalicylic acid.

16. (canceled)

17. The pharmaceutical composition according to claim 13, wherein said lozenge comprises:
a) from 1 to 10 mg of said one or more NSAIDs;
b) from 0.1 to 1 mg of lactoferrin;
c) from 1 to 10 mg of lysozyme;
c) from 10 to 100 mg of glycerol;
e) from 100 to 400 mg of sweetener;
f) from 1 to 20 mg menthol;
g) from 1 to 20 mg carboxymethyl cellulose; and
h) from 1 to 20 mg aloe.

18. The pharmaceutical composition according to claim 1, wherein said pharmaceutical composition is formulated as an oral spray.

19. A method for the treatment or prophylaxis of sore throat in a subject in need thereof comprising administering the pharmaceutical composition according to claim 1 to the subject in need thereof.

20. The method according to claim 19, wherein less than 325 mg of the plurality of NSAIDs are administered in 24 hours.

21-22. (canceled)

23. A pharmaceutical product comprising:
(a) a body configured to be inserted into an oral passage for dispensing the oral spray composition according to claim 18;
(b) a reservoir in fluid communication with said orifice, wherein said oral spray composition is contained in said reservoir;
(c) a pump mechanism capable of expelling said oral spray composition through said orifice in appropriate sized aerosolized droplets; capable of coating the oral mucosa of a user.

24. (canceled)

25. A method for the treatment or prophylaxis of sore throat in a subject in need thereof comprising:
a) inserting the portion of the pharmaceutical product according claim 23 configured to be inserted into an oral passage into the oral passage of said subject; and
b) actuating said pump mechanism to administer said oral spray composition to said subject.

26. The method according to claim 25, wherein said inserting and actuating steps are repeated at a time point more than 10 minutes after the previous of said administration oral spray composition.

27. A method for the treatment of sore throat in a subject in need thereof comprising administering less than 75 mg of a plurality of NSAIDs to said throat.

28-30. (canceled)

31. The method according to claim 19, wherein said administration has minimal or no effect on the barrier integrity of the mucosa as compared to an otherwise identical mucosa that had not been administered said one or more NSAIDs.
